(19) United States
(12) Patent Application Publication
(10) Pub. No.: US 2024/0374663 A1
(43) Pub. Date: Nov. 14, 2024

(54) METHODS AND FORMULATIONS RELATED TO THE INTRATHECAL DELIVERY OF ONCOLYTIC VIRUSES

(71) Applicant: THE UAB RESEARCH FOUNDATION, Birmingham, AL (US)

(72) Inventors: Gregory Friedman, Birmingham, AL (US); James Markert, Birmingham, AL (US); George Yancey Gillespie, Birmingham, AL (US); Joshua Bernstock, Birmingham, AL (US); Kyung-Don Kang, Birmingham, AL (US)

(73) Assignee: THE UAB RESEARCH FOUNDATION, Birmingham, AL (US)

(21) Appl. No.: 18/690,101

(22) PCT Filed: Oct. 12, 2022
(86) PCT No.: PCT/US2022/046420

§ 371 Date: Mar. 7, 2024

Related U.S. Application Data
(60) Provisional application No. 63/273,577, filed on Oct. 29, 2021.

(51) Int. Cl. A61K 35/763; A61K 9/00; A61K 31/713; A61K 38/21; A61P 35/00

(52) U.S. Cl. CPC A61K 35/763; A61K 9/0019; A61K 31/713; A61K 38/21; A61P 35/00

(57) ABSTRACT

A method of treating a central nervous system cancer in a subject comprising administering to the subject a preconditioning agent, and, administering to the subject a therapeutic dose of an oncolytic virus; wherein the preconditioning agent is an immunostimulant selected from the group consisting of a toll-like receptor agonist, a subtherapeutic dose of an oncolytic virus, and/or an interferon that induces a transient anti-viral response and/or upregulates an interferon response; and wherein the preconditioning agent and the therapeutic dose of the oncolytic virus is administered to the subject's cerebrospinal fluid or periependymal region. Provided herein are methods of treating a central nervous system cancer in a subject. This method allows for intrathecal administration of oncolytic virus, which, until the present invention, resulted in unacceptable toxicity.

### CROSS-REFERENCE TO RELATED APPLICATION

This application claims priority to Provisional Patent Application No. 63/273,577, filed Oct. 29, 2021, which is incorporated by reference herein in its entirety.

### STATEMENT AS TO RIGHTS TO INVENTIONS MADE UNDER FEDERALLY SPONSORED RESEARCH AND DEVELOPMENT

This invention was made with government support under Grant No. W81XWH-15-1-0108 awarded by the Department of Defense. The government has certain rights in the invention.

### BACKGROUND

Oncolytic viral therapy has emerged as a promising treatment for cancers. For example, immunovirotherapy with engineered oncolytic HSV-1 G207 has emerged as a promising treatment for patients with high-grade brain tumors. G207 infects and kills tumor cells while sparing normal cells and stimulates a robust anti-tumor immune response. Intratumoral G207 inoculation demonstrated safety and preliminary efficacy in clinical trials. Although intratumoral inoculation delivers G207 directly to a primary tumor, it requires invasive neurosurgical procedures, thereby limiting repeat dosing. The prevailing view is that such inoculation precludes administration of oncolytic virus intrathecally or even near the peri-ependymal space due to toxicity associated with oncolytic viruses that enter the cerebrospinal fluid. Therefore, oncolytic viral therapies that avoid toxicity associated with systemic administration and that can be administered without multiple surgical and/or using minimally intervention(s) are needed.

### BRIEF SUMMARY

Provided herein are methods of treating central nervous system (CNS) cancers. The methods include administering to a subject with a CNS cancer (primary or metastatic) a preconditioning agent that allows for delivery into cerebrospinal fluid (CSF) spaces in part via the protection of ependymal cells prior to a therapeutic dose of an oncolytic virus. The therapeutic dose of the oncolytic virus can then be administered into the subject's CSF (e.g., by injection into ventricles, subarachnoid spaces, or CSF cisterns (lumbar and the like)), to a periependymal region, and/or to a region abutting any CSF space. This method allows for administration of the oncolytic virus into or near any CSF space without the toxic effects that would result in the absence of the preconditioning step. The methods described herein provide new treatment options for CNS tumors in both children and adults and allows for delivery of agents previously considered toxic.

The details of one or more embodiments are set forth in the accompanying description below. Other features, objects, and advantages will be apparent from the description and from the claims.

### BRIEF DESCRIPTION OF THE DRAWINGS

FIGS. 1A-E show toxicity from oncolytic HSV. FIG. 1A is a graph showing time to recover body weight for CBA/J, BALB/c and C57BL/6 mice. Mice were inoculated intraventricularly (IVT) with 1×10⁷ PFU oncolytic Herpes Simplex Virus (HSV) with γ₁34.5 genes deletions and expressing murine interleukin (IL)-12, and weights were measured 3 days per week. Average time to recover body weight to baseline was calculated. CBA/J were significantly more sensitive than BALB/c and C57BL/6 mice. C57BL/6 had the least amount of toxicity. FIG. 1B shows images of hematoxylin and eosin (H&E) stained ventricular linings of CBA/J mice after G207 (an oncolytic HSV with both γ₁34.5 genes deleted in combination with insertion of lacZ into the U_{L}39 gene which encodes viral ribonucleotide reductase) inoculation. The images show focal disruption of the ependyma or ependymal lining with some nuclear shrinkage, fragmentation, and cell death at day 2. At day 4, diffuse involvement and disruption of the ependymal lining with minimal to no normal lining was apparent. Reactive ependymal proliferation was present with an influx of immune cells. Day 7-30 demonstrated a slow repair of ependymal lining. FIG. 1C are images showing HSV-1 and glycoprotein D (gD) staining in ependymal cells at Day 2. Positive gD stain seen in ependymal cells lining the ventricle indicates a productive infection in the ependymal cells. Scale bar=500 μm. Data represent mean±SD. FIG. 1D are images showing immunohistochemistry (IHC) staining for CD8+ cytotoxic T cells at day 2 after delivery of IVT saline (left) or G207 (right). Scale bar=100 μm (upper) and 25 μm (lower). FIG. 1E is a graph showing CD8+ T cell depletion experiments. CBA/J mice received anti-CD8 antibody or isotype control for two doses and then were inoculated IVT with 1×10⁷ PFU of oncolytic HSV (oHSV), and weights were measured per week. Mice deficient for CD8+ cells lost significantly less weight than mice with CD8+ cells on day 7 (*, P<0.05; **, P<0.01). Two-tailed P values were determined by unpaired t test.

FIGS. 2A-E show protection of ependymal cell lining with low dose (1×10⁴ PFU) IVT oncolytic HSV G207 or IVT poly I:C prior to high dose (1×10⁷ PFU) G207. FIG. 2A is a schematic and graph showing the effect of repeat IVT doses of oncolytic HSV. CBA/J mice were inoculated IVT with 1×10⁷ PFU of oHSV (G207) and weights were measured 3 days per week. Average total weight and percentage change from baseline were calculated on each day that weight was measured. Once mice recovered from first dose, they received a second IVT dose (1×10⁷ PFU of G207) and followed as above. This was repeated for a third dose. Mice lost significantly less weight with each subsequent dose of virus. FIG. 2B is a schematic and graph showing CBA/J mice received low dose 1×10⁴ PFU of IVT G207 or saline followed by 1×10⁷ PFU of IVT G207 and body weights were checked 2-3 times per week (N=6 mice for saline and N=7 mice for G207). Weight for each mouse was used to calculate a percentage change from baseline, and an average percentage change from baseline was compared between mice pre-treated with low dose virus versus saline. FIG. 2C is a schematic and graph showing CBA/J mice were inoculated intraventricularly with saline, 20 or 50 g of poly I:C. Three days later mice received 1×10⁷ PFU of G207 intraventricularly, and weights were measured 2-3 days per week. Average total weight and percentage change from baseline were calculated on each day that weight was measured. Weights were compared with mice that did not receive poly I:C prior to receiving the same IVT dose of G207. Mice that received poly I:C prior to virus had significantly less weight loss and recovered to baseline weight faster than mice that did not receive poly I:C prior to receiving virus. FIG. 2D is a graph showing average time to recover body weight to baseline. FIG. 2E are images showing H&E staining. Scale bar=100 μm (upper) and 25 μm (lower). HSV-1 and gD staining. Scale bar=25 μm. Data represent mean±SD (*, P<0.05; **, P<0.01; ***, P<0.001). Two-tailed P values were determined by unpaired t test.

FIGS. 3A-C show the effect of IVT Poly I:C on ependymal cell lining. CBJ/A mice were inoculated intraventricularly with saline or poly I:C 50 μg. Three days later mice received 1×10⁷ PFU of G207 intraventricularly. FIG. 3A is a schematic of the experimental protocol and images showing IHC staining of the ependymal lining performed with p-eIF2α antibody at 3, 6, or 12 h later after HSV inoculation. Scale bar=25 μm. FIG. 3B is a graph showing relative mRNA expression for TLR9 and MDA5 by RT-qPCR at day 3 after poly I:C inoculation. FIG. 3C are images showing IHC staining of the ependymal lining performed with TLR9 antibody. Scale bar=100 μm (upper) and 25 μm (lower). Data represent mean±SD (*, P<0.05).

FIGS. 4A-E show IVT G207 treatment prolongs survival and blocks spinal metastasis in athymic nude mice bearing metastatic D425-luc medulloblastoma tumors. FIG. 4A is a schematic showing mice were given D425-luc cells at day 0 and then treated with 5 μL of saline or G207 at 1×10⁶ PFU at post-tumor induction day 5 and 13. Bioluminescence imaging was performed twice weekly. FIG. 4B shows bioluminescence imaging. FIG. 4C is a graph showing Kaplan-Meier survival plots. FIG. 4D is a graph showing summary of bioluminescence staining of spinal D425-luc MB cells at days 5, 9, 12, 15, 19 and 22 post tumor induction. FIG. 4E are images showing bioluminescence of spinal D425-luc MB cells.

FIGS. 5A-E show pretreatment with low-dose oHSV prevents toxicity from IVT G207 and prolongs survival in an immunocompetent mouse model. FIG. 5A is a schematic showing athymic nude mice inoculated intraventricularly with D341 cells. Three days later mice received saline or low-dose 1×10⁴ PFU of IVT G207. Mice were treated with saline, 1×10⁶ or 1×10⁷ PFU of IVT G207 at post-tumor induction day 6 and 13. FIGS. 5B and 5C are graphs summarizing bioluminescence imaging measured in counts/second for brain (5B) and spinal cord (5C) of each mouse treated. In small graphs, quantification of the average bioluminescence signal (dark lines) and the signals for individual mice (light lines) for each group. FIG. 5D are images showing bioluminescence imaging of representative mice. FIG. 5E is a graph showing Kaplan-Meier survival plots. Data represent mean±SD (*, P<0.05, **, p<0.01). Two-tailed P values were determined by unpaired t test.

FIGS. 6A-D show pretreatment with poly I.C prevents toxicity from IVT G207 and prolongs survival in an immunocompetent mouse model. FIG. 6A is a schematic showing Albino B6 mice inoculated intraventricularly with MP tumor cells. Eleven days later mice received saline or poly I:C of IVT G207. Mice were treated with saline, 1×10⁶ or 1×10⁷ PFU of IVT G207 at post-tumor induction day 14. Bioluminescence imaging measured in counts/second. FIG. 6B are images showing bioluminescence imaging of representative mice. FIG. 6C is a graph showing a summary of bioluminescence signals for each treatment regimen. FIG. 6D is a graph showing Kaplan-Meier survival plots. Data represent mean SD (*, P<0.05; **, P<0.01). Two-tailed P values were determined by unpaired t test.

FIG. 7 is a graph showing toxicity of oncolytic HSV to CBA/J, BALB/c and C57BL/6. Mice were inoculated intraventricularly with 1×10⁷ PFU of an oncolytic HSV with γ₁34.5 gene deletions and expressing murine interleukin (IL)-12, and weights were measured 3 days per week. Average total weight and percentage change from baseline were calculated for each mouse strain on each day that weight was measured. CBA/J were significantly more sensitive than BALB/c and C57BL/6 mice. C57BL/6 had the least amount of toxicity.

FIG. 8A is an image showing IHC staining for CD8+ cytotoxic T cells at day 2 after delivery of IVT UV inactivated G207. Scale bar=200 μm. FIG. 8B are graphs showing CD8+ T cell blocking experiments. Anti-CD8 antibody and isotype control were given IP to CBA/J mice. Blocking was confirmed by flow cytometry with the antibody. There was no effect on the CD4+ T cells.

FIG. 9 is a schematic and graph showing the protection of ependymal cell lining with various low doses (1×10², 1×10³, or 1×10⁴ PFU) of IVT oncolytic HSV G207 prior to high dose (1×10⁷ PFU) G207. CBA/J mice received 1×10², 1×10³, or 1×10⁴ PFU of IVT G207s or saline followed by 1×10⁷ PFU of IVT G207 and body weights were checked 2-3 times per week (N=7 mice for saline, 1×10², or 1×10⁴ G207 and N=6 mice for G207). Average total weight and percentage change from baseline were calculated for each mouse strain on each day that weight was measured. Data represent mean±SD (*, P<0.05). Two-tailed P values were determined by unpaired t test.

FIG. 10 is a schematic and graph showing protection of ependymal cell lining with low dose (1×10⁴ PFU) IVT oncolytic HSV G207 prior to high dose (1×10⁷ PFU) G207. CBA/J mice received low dose 1×10⁴ PFU of IVT G207 or saline followed by 1×10⁷ PFU of IVT G207 and body weights were checked 2-3 times per week (N=6 mice for saline and N=7 mice for G207). Average time to recover body weight to baseline was calculated. Data represent mean±SD (**, P<0.01). Two-tailed P values were determined by unpaired t test.

FIG. 11 is a graph showing effect of low-dose pretreatment on G207. D425-luc, pediatric brain tumor xenograft cells were treated with a low Multiplicity of Infection (MOI) with G207 [0.01 or 0.001 PFU/cell]. At 72 hrs later, the cells were plated and incubated for 24 hrs. G207 was added at increasing MOI. At 72 hrs after viral inoculation, AlamarBlue assay was performed to evaluate cytotoxicity with IC₅₀ based on determination of the Intracellular Cytotoxicity for 50% of the cells within a 72 hr period (IC₅₀).

FIGS. 12A-12D shows the effect of Poly IC and IFN on G207 cytotoxicity with pediatric brain xenograft model D341-MED and D425-MED. FIG. 1A is a graph showing D341 pediatric brain tumor xenograft cells treated with G207 and/or Poly-IC. FIG. 1B is a graph showing D341 pediatric brain tumor xenograft cells treated with G207 and/or interferon (IFN). FIG. 1C is a graph showing D425 pediatric brain xenograft cells treated with G207, G207 and IFN or G207 and IFN and Poly-IC. The cells were plated and incubated for 24 hrs. Poly IC (2 g), IFN (200 IU) or Poly IC and IFN were added to the wells prior to viral inoculation. G207 was added at increasing MOI. At 72 hrs after virus inoculation, AlmarBlue assay was performed to evaluate cytotoxicity with IC50. FIG. 12D is a graph showing the effect of low-dose pretreatment on G207. D425 pediatric brain tumor xenograft cells were treated with low-dose G207 [0.01 or 0.001 MOI (PFU/cell)]. At 72 hrs later, the cells were plated and incubated for 24 hrs. G207 was added at increasing MOI. At 72 hrs after viral inoculation, AlamarBlue assay was performed to evaluate cytotoxicity with IC₅₀. The viral replication and killing of cells was not perturbed by the preconditioning agents employed.

FIG. 13 are images showing toxicity from IVT oHSV in the left lateral ventricle and third and fourth ventricles at different time points. Day 1: Intact ependymal lining without inflammatory change. Day 2: Disruption of the ependymal lining (black arrowheads) with lymphocytic inflammation (lymphocytic ependymitis). Day 4 and 7: Focal variable inflammatory infiltrate involving the ventricular lining and focal loss of lining with periventricular edema (black arrowheads). Days 16 and 30: focal loss of ventricular lining with no residual inflammation. Stretching of ependymal cells in some areas. Scale bars=100 μm.

FIG. 14 are images showing toxicity with repeated doses of IVT oHSV. Mice were injected with IVT saline or 1×10⁷ PFU of oHSV and then received a second and third dose once baseline body weight had recovered, on day 13 and 22 respectively. Mice from each group were sacrificed on days 7, 14, and 28 and brains were examined for toxicity. Representative H&E staining of the right lateral ventricular lining is shown. A normal ependymal lining was present at each time point in the saline group. Areas of disruption to the ependymal were seen one week after the first dose; however, little to no additional toxicity was seen with each successive dose. Scale bar=500 μm.

FIG. 15 is a graph showing the effect of IVT poly I:C on ependymal cell lining. Mice were inoculated with IVT saline or poly I:C 50 μg. Three days later, mice received IVT oHSV (1×10⁷ PFU). Brains and CSF were harvested at 0, 6, and 12 hours (N=3-6/group/time point) after oHSV inoculation, and IFN-β was measured by ELISA. Significance was calculated using a two-way ANOVA with Sidik's multiple comparison test (**** P<0.0001).

FIG. 16 is a graph showing interferon β (IFN β) levels over time in the CSF after a single 50 μg IVT dose of poly I:C or saline (N=2 mice/group/time point).

FIGS. 17A, 17B, 17C and 17D are graphs showing effects of poly I:C alone and combined with oHSV in vitro. Poly I:C (0 to 100 μg/mL for 72 hours) had no inhibitory effects on D341 cells (17A) or MP cells (17B). Poly I:C did not enhance G207 cytotoxicity of tumor cells when given 24 hours prior to or at the same time as G207 in D341 (17C) cells or MP cells (17D). Significance was calculated using two-way ANOVA with Tukey's multiple comparison test. Data represent mean±SD (*, P<0.05).

### DETAILED DESCRIPTION

Primary malignant brain tumors and extracranial solid tumor brain metastases in adults and children are associated with severe disability and poor outcomes, requiring less-toxic, more effective therapies to improve outcomes. Immunotherapy with intratumoral oncolytic HSV (oHSV) has shown promise in clinical trials. Direct intratumoral inoculation delivers concentrated virus directly to tumor cells but requires an invasive neurosurgical procedure that limits repeat injections and precludes direct targeting of metastatic and leptomeningeal disease. Intraventricular delivery of oHSV would overcome these limitations but has been avoided due to toxicity concerns. Herein, toxicity from intraventricular oHSV is shown to be mitigated with protective pretreatment approaches that enabled effective treatment of disseminated brain tumor models with oHSV G207, an attenuated oHSV, thus showing preclinical safety and efficacy of intraventricular G207.

Central nervous system (CNS) tumors are the most common pediatric solid tumors and the leading cause of childhood cancer-related morbidity and mortality. Likewise, adult primary malignant brain tumors and extracranial solid tumor metastases to the brain are associated with severe disability and poor outcomes. Leptomeningeal metastatic disease (LMD) occurs in 30-50% of newly diagnosed and recurrent pediatric malignant cerebellar tumors, 20-45% of malignant supratentorial tumors and 5-25% of adult solid tumors, including approximately 20% of glioblastomas. Radiation and chemotherapy often cause substantial long-term neurotoxicity and outcomes remain poor for pediatric patients with LMD. At recurrence, LMD is generally minimally responsive to conventional therapies. LMD is also a significant problem in adult diseases (i.e., both primary and metastatic to the CNS). Immunovirotherapy with engineered oncolytic HSV-1 G207 has emerged as a promising treatment for high-grade brain tumors. While intratumoral inoculation delivers G207 directly to a primary tumor, it requires neurosurgical procedures thereby limiting repeat doses. The present invention establishes the safety and efficacy of intrathecal/intraventricular administration of oncolytic viruses, e.g., G207, for introduction of oncolytic virus to the CSF.

In addition to LIMD, medulloblastoma is a common malignant brain tumor in children that can be challenging to successfully treat. Oncolytic herpes simplex virus-1 (oHSV) is a promising therapy for a myriad of solid pediatric neoplasms, but optimal delivery of these viruses to the central nervous system remains challenging. Given that medulloblastomas arise in the cerebellum or fourth ventricle, patients often have disseminated metastases throughout the brain and spinal cord. As described in the examples below, a clinical grade engineered oHSV, G207, was tested in intraventricular models. Pretreatment using polyinosinic-polycytidylic acid (poly I.C), a toll-like receptor 3 agonist, or low-dose oHSV prevented damage to the ependymal lining of ventricles. This approach enabled safe delivery of multiple subsequent therapeutic doses of G207. Importantly, G207 significantly prolonged survival in a pediatric patient-derived xenograft model and an immunocompetent murine model of medulloblastoma. Such results show that the toxicity from intraventricular G207 can be safely mitigated and that intraventricular G207 effectively targets medulloblastoma metastases. These findings for the first time show that intraventricular injection of oHSV is a promising route of delivery for the treatment of medulloblastoma and other cancers throughout the brain and spine.

Further, as demonstrated herein, a standard dose of intraventricular (IVT) oHSV damaged the ependymal lining in an immunocompetent, HSV-sensitive murine strain, but the ependyma was protected with IVT low-dose oHSV or polyinosinic-polycytidylic acid (poly I:C) prior to an IVT treatment dose of oHSV. This approach with clinically relevant oHSV G207 enabled safe delivery of multiple therapeutic doses and prolonged survival in human and murine metastatic medulloblastoma models. These findings indicate that toxicity from IVT oHSV can be mitigated, allowing for a therapeutic approach to targeting disseminated, LMD.

Thus, provided herein are methods of treating a central nervous system (CNS) cancer in a subject. The methods include administering to the subject a preconditioning agent, and, then, administering to the subject a therapeutic dose of an oncolytic virus. Optionally, the preconditioning agent and/or the oncolytic virus are administered to the subject's cerebrospinal fluid (CSF), to a periependymal region, and/or to a region abutting a CSF space (including, for example, a resection cavity). Without meaning to be limited by theory, the preconditioning agent is thought to stimulate protection of ependymal cells such that the subsequent therapeutic dose of the oncolytic virus is not unacceptably toxic.

As used herein, a CNS cancer can be any cancer in the brain or spinal cord including, for example, astrocytomas, oligodendrogliomas, gliomas, ependymal tumors, medulloblastomas, pineal parenchymal tumors, meningeal tumors, germ cell tumors, and craniopharyngioma. The CNS cancer can be a cancer that has metastasized to the CNS. Optionally, the central nervous system cancer treated by the disclosed methods include any periventricular, intraventricular, cisternal, or intracisternal cancer or any cancer in the CNS abutting a CSF space, including a metastasis (e.g., a drop metastasis) or a leptomeningeal cancer, which were not previously treatable by intratumoral injection of oncolytic viruses. However, the methods are useful in treating all forms of CNS cancer.

Optionally, the preconditioning agents and/or the oncolytic virus can be administered intrathecally (including, for example, intraventricularly or intracisternally). Optionally, the preconditioning agents and/or oncolytic virus is administered into the subarachnoid space, for example, into the lumbar cistern. The preconditioning agents and/or oncolytic virus can also be administered to a lesion adjacent to or near a cerebral spinal fluid containing space, such as a resection cavity. Optionally, the preconditioning agents and/or oncolytic virus are administered periventricularly or periependymally, intraventricularly, intracisternally, or contiguous with a cerebral spinal fluid containing space.

Preconditioning agents can be an immunostimulants and/or agents that induce a transient anti-viral response and/or agents upregulating an interferon response. Optionally, an immunostimulant is selected from the group consisting of a toll-like receptor agonist (e.g., Poly-IC; Poly-IC:LC; or a fragment of repetitive unmethylated CpG nucleic acid sequence (RNA or DNA)), a subtherapeutic dose of an oncolytic virus, and/or an interferon or an interferon stimulating agent (e.g., RIG-1 or MDA5 agonists, interleukins, tumor necrosis factor (TNF) and the like).

Optionally, the preconditioning agent is provided as a protective dose (referred to herein as a subtherapeutic dose) of an oncolytic virus. The oncolytic virus administered as the preconditioning agent can be the same oncolytic virus subsequently administered at the therapeutic dosing step. The virus used as a preconditioning dose can be a genetically modified virus, an attenuated virus, an inactivated virus, a dead virus or a fragment thereof so long as the virus used as a preconditioning agent provides a protective function.

Oncolytic viruses that are used in the provided methods and kits include, but are not limited to, oncolytic viruses that are members in the family of herpesviridae, myoviridae, siphoviridae, podpviridae, teciviridae, corticoviridae, plasmaviridae, lipothrixviridae, fuselloviridae, poxviridae, iridoviridae, phycodnaviridae, baculoviridae, adenoviridae, papovaviridae, polydnaviridae, inoviridae, microviridae, geminiviridae, circoviridae, parvoviridae, hepadnaviridae, retroviridae, cyctoviridae, reoviridae, birnaviridae, paramyxoviridae, rhabdoviridae, filoviridae, orthomyxoviridae, bunyaviridae, arenaviridae, leviviridae, picornaviridae, sequiviridae, comoviridae, potyviridae, caliciviridae, astroviridae, nodaviridae, tetraviridae, tombusviridae, coronaviridae, glaviviridae, togaviridae, and barnaviridae. Immunoprotected viruses and reassortant or recombinant viruses of these and other oncolytic viruses are also encompassed by the provided methods. The oncolytic virus used in the provided methods is, optionally, a chimeric virus, an armed virus, and/or a modified virus. By way of example, the oncolytic virus can be a herpes simplex virus, a chimeric herpes simplex virus (e.g., C134), an armed herpes simplex virus (e.g., a cytokine-armed herpes virus, M032), or a genetically modified herpes simplex virus. Furthermore, a combination of at least two (or more) oncolytic viruses can also be employed to practice the provided methods. For example, the preconditioning agent can be a virus that induces an interferon response (e.g., measles virus) and the oncolytic virus can be a different virus, e.g., an oncolytic HSV. A few oncolytic viruses are discussed below, and a person of ordinary skill in the art can practice the present methods using additional oncolytic viruses as well according to the disclosure herein and knowledge available in the art. Optionally, the methods include the use of oncolytic viruses with mutations including (insertions, substitutions, deletions, rearrangements or duplications) in one or more genome segments. Such mutations can comprise additional genetic information as a result of recombination with a host cell genome or can comprise synthetic genes, such as, for example, genes encoding agents that suppress anti-viral immune responses.

A mutation as referred to herein can be a substitution, insertion or deletion of one or more nucleotides. Point mutations include, for example, single nucleotide transitions (purine to purine or pyrimidine to pyrimidine) or transversions (purine to pyrimidine or vice versa) and single- or multiple-nucleotide deletions or insertions. A mutation in a nucleic acid can result in one or more conservative or non-conservative amino acid substitutions in the encoded polypeptide, which may result in conformational changes or loss or partial loss of function, a shift in the reading frame of translation (frame-shift) resulting in an entirely different polypeptide encoded from that point on, a premature stop codon resulting in a truncated polypeptide (truncation), or a mutation in a virus nucleic acid may not change the encoded polypeptide at all (silent or nonsense). See, for example, Johnson and Overington, 1993, J. Mol. Biol. 233:716-38; Henikoff and Henikoff, 1992, Proc. Natl. Acad. Sci. USA 89:10915-19; and U.S. Pat. No. 4,554,101, for disclosure on conservative and non-conservative amino acid substitutions.

Mutations can be generated in the nucleic acid of an oncolytic virus using any number of methods known in the art. One of the most common methods of site-directed mutagenesis is oligonucleotide-directed mutagenesis. In oligonucleotide-directed mutagenesis, an oligonucleotide encoding the desired change(s) in sequence is annealed to one strand of the DNA of interest and serves as a primer for initiation of DNA synthesis. In this manner, the oligonucleotide containing the sequence change is incorporated into the newly synthesized strand. See, for example, Kunkel, 1985, Proc. Natl. Acad. Sci. USA 82:488; Kunkel et al., 1987, Meth. Enzymol. 154:367; Lewis and Thompson, 1990, Nucl. Acids Res. 18:3439; Bohnsack, 1996, Meth. Mol. Biol. 57:1; Deng and Nickoloff, 1992, Anal. Biochem. 200:81; and Shimada, 1996, Meth. Mol. Biol. 57:157. Other methods are used routinely in the art to modify the sequence of a protein or polypeptide. For example, nucleic acids containing a mutation can be generated using PCR or chemical synthesis, or polypeptides having the desired change in amino acid sequence can be chemically synthesized. See, for example, Bang and Kent, 2005, Proc. Natl. Acad. Sci. USA 102:5014-9 and references therein.

Optionally, the oncolytic virus is an oncolytic herpes simplex virus. A herpes simplex virus 1 (HSV-1) mutant defective in ribonucleotide reductase expression, hrR3, replicates in colon carcinoma cells but not normal liver cells (Yoon et al., FASEB J. 14:301-311(2000)). Optionally, the oncolytic virus is a replication-competent, recombinant herpes simplex virus-1 (HSV-1). Optionally, the HSV-1 comprises a mutation in 734.5 gene and an inactivating mutation in ICP6 gene. The mutation in the ICP6 gene comprises an insertion of the E. coli lacZ gene. Optionally, the mutation can be a combination of these named mutations and others including the insertion of complementary DNA from a closely related alphaherpesvirus (HSV-2) or a more distantly related betaherpesvirus (human Cytomegalovirus; HCMV). Optionally, the herpes simplex virus is R7017 or R7020 (also known as NV1020) (Meignier B, Longnecker R, Roizman B. In vivo behavior of genetically engineered herpes simplex viruses R7017 and R7020: construction and evaluation in rodents. J Infect Dis. 1988 September; 158(3):602-14). Optionally, the herpes simplex virus is C134 (Shah A C, et al., Enhanced antiglioma activity of chimeric HCMV/HSV-1 oncolytic viruses. Gene Ther. 2007 July; 14(13):1045-54). The oncolytic HSV is optionally G207. G207 contains deletion of the diploid 7134.5 neurovirulence gene and has viral ribonucleotide reductase (UL39) disabled by insertion of Escherichia coli lacZ. G207 is described in Mineta et al., Attenuated multi-mutated herpes simplex virus-1 for the treatment of malignant gliomas. Nat Med 1995; 1:938-43, which is incorporated herein by reference in its entirety. Optionally, the genetically modified herpes simplex virus is IMILYGIC® (Amgen, Thousand Oaks, CA).

Typically, for oncolytic viruses, approximately 1 to 1×10¹⁵ plaque forming units (PFU) of a virus are used, depending on the type, size and number of proliferating cells or neoplasms present. The effective amount can be, for example, from about 1×10² PFU to about 1×10¹⁵ PFU). Optionally, the effective amount is about 1×10⁸ to about 1×10¹² PFU or TCID₅₀. Optionally, the effective amount is about 1×10³ to about 1×10⁹ PFU or TCID₅₀. Optionally, the effective amount is about 3×10¹⁰ to about 1×10¹⁰ TCID₅₀. The method can include administering one or more additional therapeutic doses of the oncolytic virus.

As used herein, a subtherapeutic dose of an oncolytic virus refers to a protective dose and is not mean to imply the complete absence of therapeutic effects. Rather, the subtherapeutic dose is any protective dose lower than the selected therapeutic dose. For example, the subtherapeutic dose can be a dose of the oncolytic virus that is at least 1×10¹ PFUs less than the therapeutic dose of the oncolytic virus. Thus, for example, if the therapeutic dose of oncolytic virus is 1×10³ to 1×10⁹ PFU, a subtherapeutic dose of the oncolytic virus would be at least 1×10¹ PFU less than the dose selected in this range (i.e., 1×10² to 1×10⁸). Thus, for example, if the therapeutic dose is 1×10⁷ the subtherapeutic dose would be 1×10⁶ or less, e.g., 1×10⁵, 1×10⁴, 1×10³, 1×10², or 1×10¹ PFU) or any amount between the recited PFU amounts.

As provided herein, the preconditioning agent is administered prior to the oncolytic virus, as a primer. One of skill in the art would determine how soon after injection of the preconditioning agent the therapeutic dose of oncolytic virus should be provided. The selected time will be long enough to allow for immunostimulation, a transient anti-viral response, and/or an upregulation of interferon mediated signaling (e.g., so as to provide protection of the ependymal cells) but not so long as to unnecessarily delay treatment with the therapeutic dose of the oncolytic virus or to allow the protective effect to wane. For example, the preconditioning agent can precede the therapeutic dose of the oncolytic virus by hours or days. Optionally, the preconditioning agent can precede the therapeutic dose of the oncolytic virus by 24 to 72 hours or by 4, 5, or more days. Furthermore, such treatments (preconditioning followed by oncolytic viral therapy) can be repeated as necessary.

Optimal dosages of preconditioning agents, oncolytic viruses and other therapeutic agents, or compositions comprising the agents or viruses depend on a variety of factors. The exact amount and frequency required will vary from subject to subject, depending on the species, age, weight and general condition of the subject, the severity of the disease being treated, the particular agents used and its mode of administration. Thus, it is not possible to specify an exact amount for every composition. An appropriate amount can be determined by one of ordinary skill in the art using routine experimentation given the guidance provided herein. Effective dosages and schedules for administering the treatment regimens may be determined empirically. For example, animal models for a variety of proliferative disorders can be developed or obtained commercially (e.g., from Jackson Laboratory, 600 Main Street, Bar Harbor, Maine 04609 USA). Both direct (e.g., histology or imaging of tumors) and functional measurements (e.g., survival of a subject, size of a tumor, body weight) can be used to monitor response to therapies. These methods involve the sacrifice of representative animals to evaluate the population, increasing the animal numbers as necessary for the experiments. Measurement of luciferase activity in the tumor provides an alternative method to evaluate tumor volume without animal sacrifice and allowing longitudinal population-based analysis of therapy.

Dosage ranges for the administration of compositions are those large enough to produce the desired effect in which the symptoms of the disease are affected. The dosage should not be so large as to cause adverse side effects, such as unwanted cross-reactions, anaphylactic reactions, or cytokine storms. The dosage can be adjusted by the individual physician in the event of any counter-indications. Thus, dosages vary and are administered in one or more dose administrations, for example, daily, for one or several days. The provided agents are administered in a single dose or in multiple doses (e.g., two, three, four, six, or more doses). For example, where the administration is by infusion, the infusion can be a single sustained dose or can be delivered by multiple infusions. Treatment may last from several days to several months or until diminution of the disease is achieved.

Provided herein are pharmaceutical compositions comprising the preconditioning agents or the oncolytic viruses. The herein provided compositions are administered in a pharmaceutically acceptable carrier. A pharmaceutically acceptable carrier can be a solid, semi-solid, or liquid material that can act as a vehicle, carrier or medium for the oncolytic virus or preconditioning agent. By way of example, oncolytic viruses can be carried by cells, such as mesenchymal stem cells, which home to tumors, or genetically modified cells that target tumor cells. Accordingly, the oncolytic virus can be administered by administering the cells containing the virus.

Optionally, the compositions containing an oncolytic virus or preconditioning agent are suitable for infusion into the CSF, for example, through a catheter into or near a CSF space such as the lumbar cistern. For example, two types of fluids that are commonly used are crystalloids and colloids. Crystalloids are aqueous solutions of mineral salts or other water-soluble molecules. Colloids contain larger insoluble molecules, such as gelatin; blood itself is a colloid. The most commonly used crystalloid fluid is normal saline, a solution of sodium chloride at 0.9% concentration, which is close to the concentration in the blood (isotonic). Ringer's lactate or Ringer's acetate is another isotonic solution often used for large-volume fluid replacement. A solution of 5% dextrose in water, sometimes called D5W, is often used instead if the patient is at risk for having low blood sugar or high sodium.

Suitable carriers include phosphate-buffered saline or another physiologically acceptable buffer, lactose, dextrose, sucrose, sorbitol, mannitol, starches, gum acacia, calcium phosphate, alginates, tragacanth, gelatin, calcium silicate, microcrystalline cellulose, polyvinylpyrrolidone, cellulose, sterile water, syrup, and methyl cellulose.

The provided methods may be further combined with other tumor therapies such as radiotherapy, surgery, hormone therapy, immunotherapy, and/or adoptive cell therapy for delivery of a therapeutic payload. Thus, the provided methods can further include administering one or more additional therapeutic agents to the subject. Optionally, the therapeutic agent is one or more chemotherapeutic agents (e.g., anti-proliferative agents or anti-metabolite agents). Chemotherapeutic agents include, but are not limited to, alkylating agents, anthracyclines, taxanes, epothilones, histone deacetylase inhibitors, inhibitors of Topoisomerase I, inhibitors of Topoisomerase II, kinase inhibitors, nucleotide analogs and precursor analogs, peptide antibiotics, platinum-based compounds, retinoids, and vinca alkaloids and derivatives. Chemotherapeutic agents also include biologic agents. Chemotherapeutic agents can be delivered as a prodrug or as an active agent.

The provided methods can also include measuring or screening for tumor growth or metastasis. The screening or measuring methods can be used to determine if the oncolytic viruses and/or agents are treating the cancer. A proliferative disorder is treated when administration of the preconditioning agents and oncolytic viruses to proliferating cells results in a reduction in the number of abnormally proliferating cells, a reduction in the size of a neoplasm, and/or a reduction in or elimination of symptoms (e.g., pain) associated with the proliferating disorder. The reduction can be determined, for example, by measuring the reduction in the size of a neoplasm or in the tumor DNA present in a biological sample from the subject (e.g., a sample of CSF).

As used herein, the term subject can be a vertebrate, more specifically a mammal (e.g., a human, horse, pig, rabbit, dog, sheep, goat, non-human primate, cow, cat, guinea pig or rodent), a fish, a bird or a reptile or an amphibian. The term does not denote a particular age or sex. Thus, adult and newborn subjects, whether male or female, are intended to be covered. As used herein, patient or subject may be used interchangeably and can refer to a subject with a disease or disorder. The term patient or subject includes human and veterinary subjects.

Disclosed are materials, compositions, and components that can be used for, can be used in conjunction with, can be used in preparation for, or are products of the disclosed methods and compositions. These and other materials are disclosed herein, and it is understood that when combinations, subsets, interactions, groups, etc. of these materials are disclosed that while specific reference of each various individual and collective combinations and permutation of these compounds may not be explicitly disclosed, each is specifically contemplated and described herein. For example, if an inhibitor is disclosed and discussed and a number of modifications that can be made to a number of molecules including the inhibitor are discussed, each and every combination and permutation of the inhibitor, and the modifications that are possible are specifically contemplated unless specifically indicated to the contrary. Likewise, any subset or combination of these is also specifically contemplated and disclosed. This concept applies to all aspects of this disclosure including, but not limited to, steps in methods of using the disclosed compositions. Thus, if there are a variety of additional steps that can be performed it is understood that each of these additional steps can be performed with any specific method steps or combination of method steps of the disclosed methods, and that each such combination or subset of combinations is specifically contemplated and should be considered disclosed.

Throughout this application, various publications are referenced. The disclosures of these publications in their entireties are hereby incorporated by reference into this application.

A number of aspects have been described. Nevertheless, it will be understood that various modifications may be made. Furthermore, when one characteristic or step is described it can be combined with any other characteristic or step herein even if the combination is not explicitly stated. Accordingly, other aspects are within the scope of the claims.

### EXAMPLES

### Example 1. Safety and Efficacy of Intraventricular Immunovirotherapy with Oncolytic Virus for the Treatment of Malignant Tumors of the Central Nervous System

CBA/J lost more body weight than other mouse strains such as BALB/c, C57BL/6, and athymic nude mice, and 33% mice died after 1×10⁷ PFU of oHSV. Using this strain, methods to protect the ependymal lining from IVT G207 were developed as described herein. Low-dose treatment can prevent weight loss, and all mice survived the experiment. Therefore, low-dose pre-treatment prolongs the survival of the mice, likely by preventing ependymal cell damage.

Another option to prevent ependymal cell lining is to induce production of IFN in cells prior to giving virotherapy by treating with TLR agonists. Pretreatment with poly I:C induced early expression of several immune response genes in the brain and resulted in HSV-1 resistance. Pretreatment with IVT poly I:C prevented the loss of body weight and mediated the protective effect on ependymal cell lining. Poly I:C was protective against oHSV even after administration of the virus at 48 hours and 72 hours later.

Thus, low dose oHSV and poly I:C treatment prior to IVT oHSV reduces the degree of disruption of the ependymal lining and the associated weight loss. As for the efficacy of IVT oHSV delivery, mice receiving an IVT injection of G207 exhibited a clear reduction in the growth of tumors generated from human medulloblastoma cell lines. G207-treated mice, differing from control mice, also exhibited no detectable spinal metastases. Overall, G207-treated mice experienced a significant increase in their mean survival time.

### Materials and Methods

### Animal Studies

Animal studies were approved by the Institutional Animal Care and Use Committee (IACUC protocol APN10299) at the University of Alabama at Birmingham and were performed in accordance with all relevant NIH guidelines. BALB/c (RRID:IMSR_CRL:028), CBA/J (RRID:IMSR_JAX:000656), C57BL/6 (RRID:IMSR_CRL:027), albino B6 (RRID:IMSR_JAX:000058) and athymic nude mice (RRID:IMSR_CRL:490), ages 4-6 weeks, were purchased from The Jackson Laboratory (Bar Harbor, ME) or Charles River (Wilmington, MA).

### Patient-Derived Medulloblastoma Xenografts and Mouse Cell Lines

D341-luc and D425-luc, molecular group 3 medulloblastoma with high MYC amplification, were established from pediatric patients and were provided by Darell D. Bigner, M. D., Duke University Medical Center (He X M, et al. J Neuropathol Exp Neurol. 1989; 48(1):48-68; He et al. Lab Invest. 1991; 64(6):833-43.). The xenografts were maintained via serial transplantation in athymic nude mice as has been previously described (Friedman G K, et al. Enhanced Sensitivity of Patient-Derived Pediatric High-Grade Brain Tumor Xenografts to Oncolytic HSV-1 Virotherapy Correlates with Nectin-1 Expression Sci Rep. 2018 Sep. 17; 8(1):13930. doi: 10.1038/s41598-018-32353-x. PMID: 30224769). Authentication of human cell lines was determined by Short Tandem Repeat (STR) profiling performed by the UAB Heflin Center for Genomic Science.

MP tumor cells were generated via infection with Myc-IRES-Luciferase and DNp53-IRES-GFP retroviruses and were stereotactically injected into the cerebellum of mice. The cells were provided by Robert J Wechsler-Reya, M. D., Sanford Burnham Prebys. Medical Discovery Institute (Pei Y, et al. Cancer Cell. 2012; 21(2):155-67). 9728 is a murine medulloblastoma MYC-overexpressed group 3 (non-SHH/non-WTN) cell line provided by Martine Roussel, St. Jude Children's Hospital (Bernstock J D, et al. Cancer Gene Ther. 27(3-4):246-255 (2020)).

### Cytotoxicity Assay

Cells were dissociated using Accutase (Innovative Cell Technologies, Inc., San Diego, CA) and plated at 1×10⁴ cells/well in 96 well plates. After overnight culture, graded doses from 0 to 3.3 plaque forming units (PFU) per cell of G207 were added to each row and cytotoxicity was measured at 72 hours post-infection with alamarBlue (Life Technologies, Grand Island, NY) as previously described (Friedman G K, et al. Sci Rep. 2018; 8(1):13930; Friedman G K, et al. Gene Ther. 2015; 22(4):348-55.). Poly I:C (2 μg) was added to the wells at 24 hours prior to viral inoculation. One hour prior to viral inoculation, another 2 μg of Poly I:C was added to the wells. Graded doses of the virus were internally compared to mock-treated controls, which were included with each experiment and represented 100% tumor cell survival. Color changes of alamarBlue were quantified with a BioTek microplate spectrophotometer (Winooski, VT), and OD 595 nm values were used to calculate the IC₅₀.

Genetically Engineered Oncolytic Herpes Simplex Virus (oHSV) G207

G207 has been previously described (Mineta T, et al., Attenuated multi-mutated herpes simplex virus-1 for the treatment of malignant gliomas. Nat Med. 1995; 1(9):938-43.); briefly, G207 was engineered with deletion of both copies of the 7134.5 gene and insertion of lacZ into the U_{L}39 gene which encodes viral ribonucleotide reductase.

### Immunohistochemical (IHC) Analyses

Mouse brains were fixed in a 10% formalin solution for 24 hours (Fisher Scientific, Pittsburgh, PA) and subsequently stored in 70% ethanol. All procedures involved in the immunohistochemical (IHC) staining and hematoxylin & eosin staining have been described previously (Bernstock J D, et al. Safety and efficacy of oncolytic HSV-1 G207 inoculated into the cerebellum of mice. Cancer Gene Ther. 2020; 27(3-4):246-55). Briefly, tissue sections were cut with a microtome (6 μm). Slides were incubated overnight at 4° C. with primary antibodies including CD8 (Bioss), HSV-1 (Abcam), gD (Santa Cruz), TLR9 (Novus), and p-eIF2α (Cell Signaling). The following day, slides were incubated with OneStep Polymer horseradish peroxidase (HRP) anti-rabbit or mouse (GeneTex) for 60 minutes at room temperature. Slides were incubated with substrate 3, 3′-diaminobenzidine (DAB) (BioGenex, Fermont, CA) for 1-5 minutes and counterstained with hematoxylin (Fisher Scientific).

### CD8 Inhibition

CBA/J mice received an anti-CD8 antibody (BioXcell) or isotype control for two doses and then were inoculated IVT with 1×10⁷ PFU of G207. Weights were measured each week and mice continued to receive anti-CD8 antibody or control twice weekly. To confirm the inhibition of CD8⁺ cells, blood from the mice was collected and stained with CD4 (eBioscience), CD8 (BioLegend). Samples were then analyzed using a ThermoFisher Attune NxT Flow Cytometer (UAB Flow Cytometry Core Facility). Data were processed using FlowJo software V10 (Tristar).

qRT-PCR Analysis

CBA/J mice were injected IVT with 50 μg poly IC or saline and sacrificed 3 days after injection. RNA extraction was performed using RNeasy Plus Mini Kit (Qiagen, Germantown, MD) as described by the manufacturer. RNA quantity and purity were determined using a NanoDrop 2000 Spectrophotometer (ThermoFisher Scientific, Charlotte, NC). Complementary DNA (cDNA) was generated from 200 ng of total RNA, using High-Capacity RNA-to-cDNA Kit (ThermoFisher Scientific, Charlotte, NC). Quantitative real-time PCR was performed using TaqMan Gene Expression Assays (Applied Biosystems) for TLR9 and MDA5 on an Applied Biosystems QuantStudio 6. Results were normalized to GAPDH expression and analyzed relative to their control counterparts. The relative quantification of the gene of interest was determined using the comparative CT method (2^{-ΔΔCt}).

### In Vivo Toxicity Studies

To evaluate toxicity related to IVT delivery, mice were injected with 1×10⁷ PFU of G207 using the following stereotactic coordinates: 1 mm to the right and 0.3 mm anterior to the bregma, and 2.5 mm below the skull surface. Mice were assessed daily for signs of toxicity and had body weights measured 3 times per week. CBA/J mice were inoculated IVT with saline, poly IC (1 mg/kg or 2.5 mg/kg) or low dose of G207 (1×10⁴ PFU). Three days later, mice received 1×10⁷ PFU of G207 and weights were measured twice per week.

### In Vivo Efficacy Studies

To investigate the efficacy of IVT injection, athymic nude mice were stereotactically injected with 2.5×10⁵ D341 or D425 cells intraventrically. After 5 days and 12 days, mice were stereotactically IVT inoculated with 1×10⁶ PFU of G207 or saline. Mice were assessed daily for toxicity. Bioluminescence imaging was performed twice per week. Moribund mice were euthanized, and the date of death was recorded.

### Bioluminescence Imaging (BLI)

Mice were anesthetized with isoflurane and firefly D-luciferin potassium salt stock solution was injected IP into the mice. Imaging was performed in anesthetized mice using an IVIS 100 charge-coupled device imaging system (Xenogen, Alameda, CA). Imaging data were analyzed using Living Image Version 3.2 software. Bioluminescence intensity regions of interest (ROIs) are displayed in photons mode (counts/second) and were compared using the same BLI intensity scale and setting in both saline and oHSV treatment groups.

### Statistical Analyses

Statistical analyses were performed using GraphPad Prism (Version 5). Survival curves were created using Kaplan-Meier. Survival between groups was compared using the long-rank test. A p-value of 0.05 was considered statistically significant.

### Results

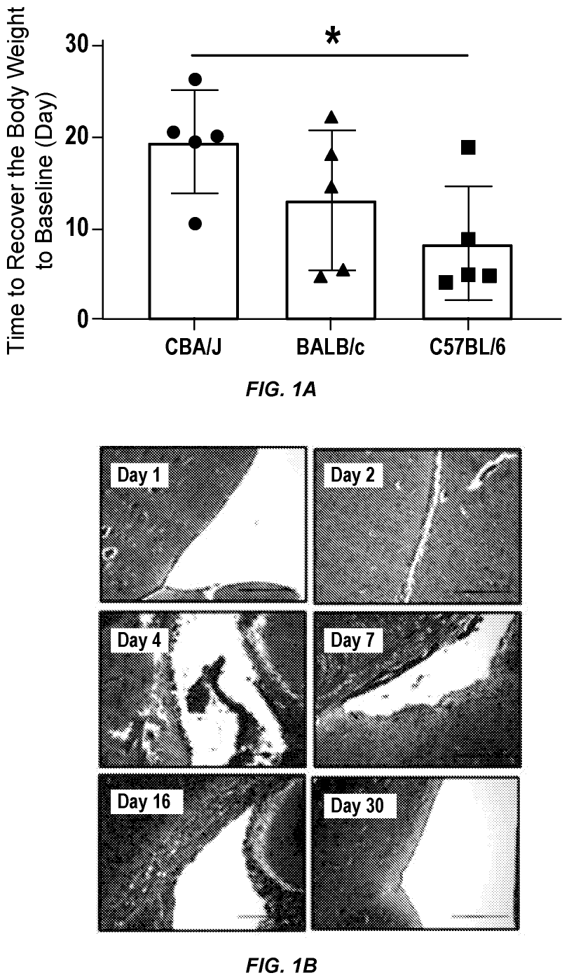

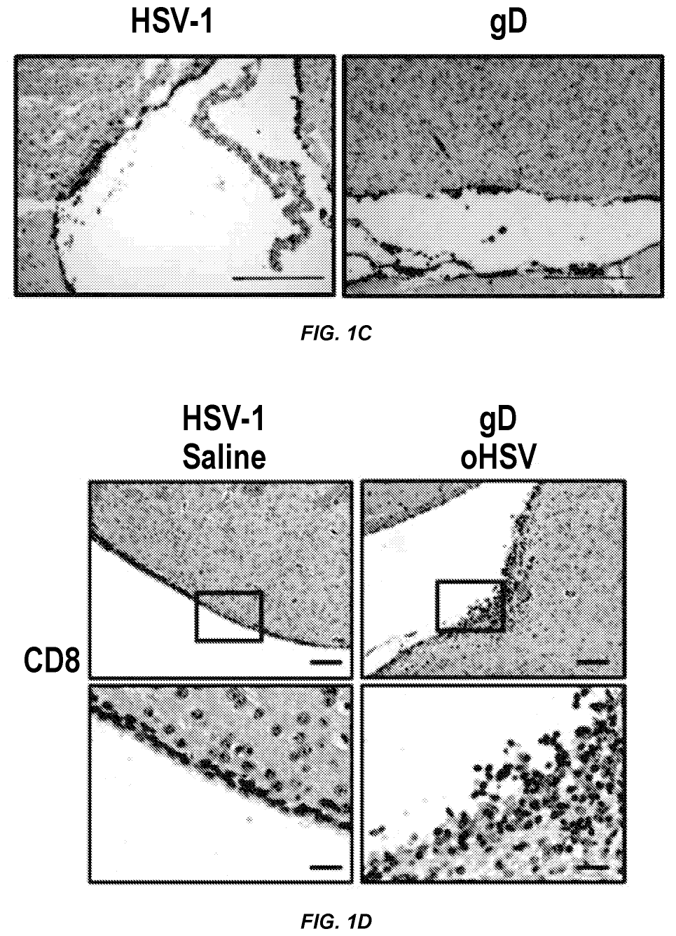

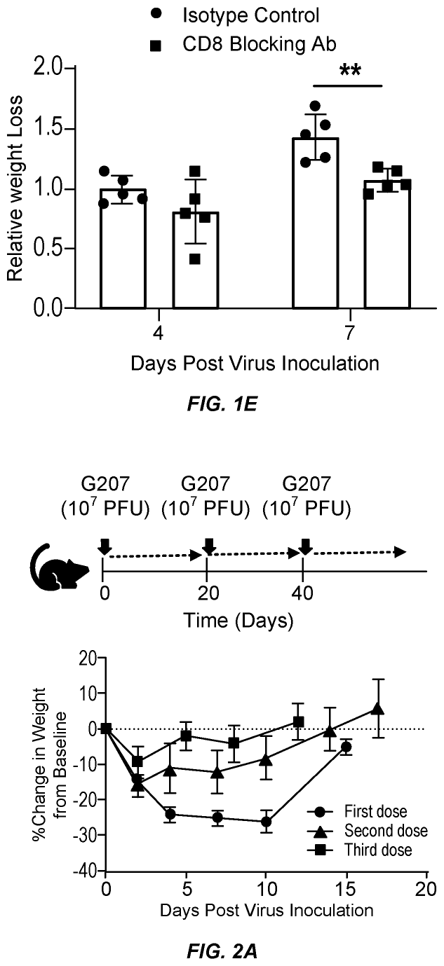

IVT delivery of G207 damages the ependymal lining of the lateral ventricles in CBA/J mice Previous research has shown that C57BL/6 mice are resistant to infection with HSV whereas other strains such as BALB/c, DBA/2, and CBA/J are highly susceptible (Zawatzky R, et al., J Infect Dis. 1982; 146(3):405-10; Kastrukoff L F, et al., Herpesviridae. 2012; 3:4; Cassady K A, et al. Mol Ther Oncolytics. 2017; 5:1-10). Accordingly, three different mouse strains were employed to determine their sensitivity to IVT G207. As evidenced by the percent change in weight from baseline (i.e., an accepted marker for toxicity (Talbot S R, et al., Defining body-weight reduction as a humane endpoint: a critical appraisal. Lab Anim. 2020 February; 54(1):99-110. doi: 10.1177/0023677219883319. Epub 2019 Oct. 30. PMID: 31665969), CBA/J mice were the most sensitive to IVT G207, whereas BALB/c displayed intermediate sensitivity and whereas C57BL/6 mice were essentially unaffected (FIGS. 1A and 7). CBA/J mice lost more body weight and required 19.5±5.7 days compared to 8.2 5.8 days to recover body weight; BALB/c mice required 13.1±7.6 days to recover body weight. Given these data the CBA/J model was employed for additional studies to determine the mechanism(s) underlying such IVT oHSV-induced toxicity.

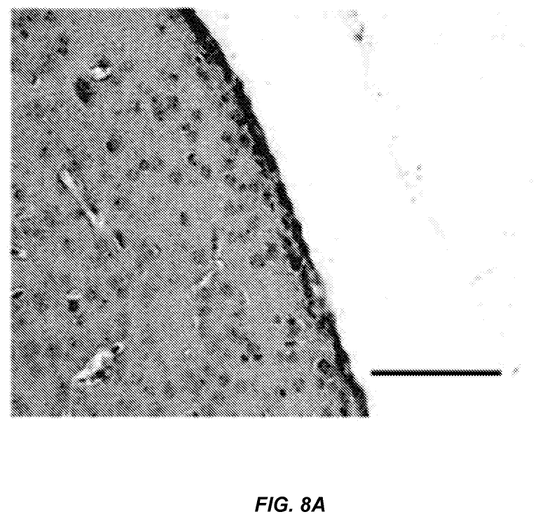

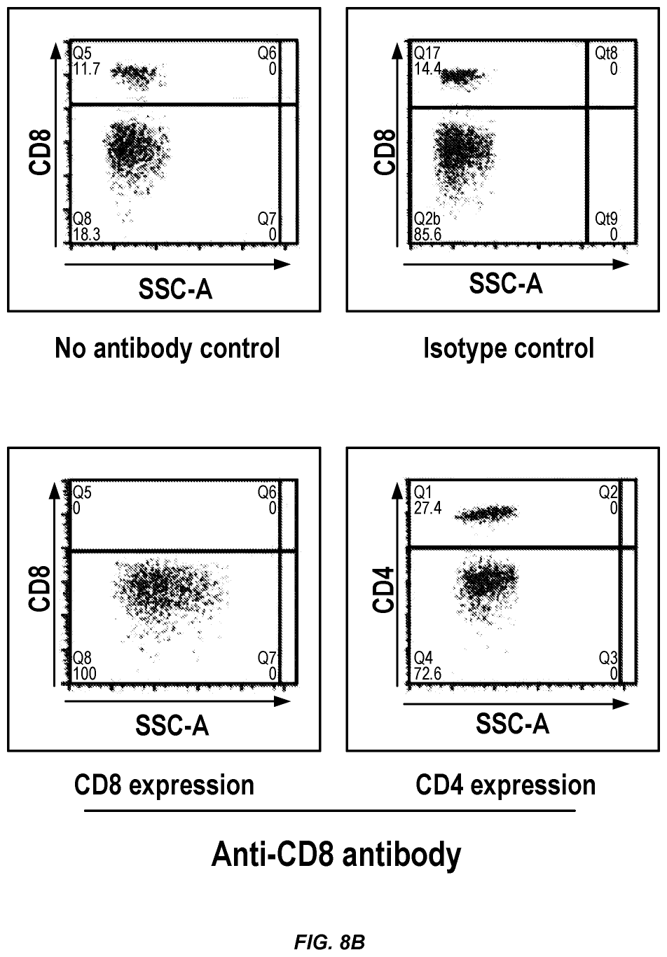

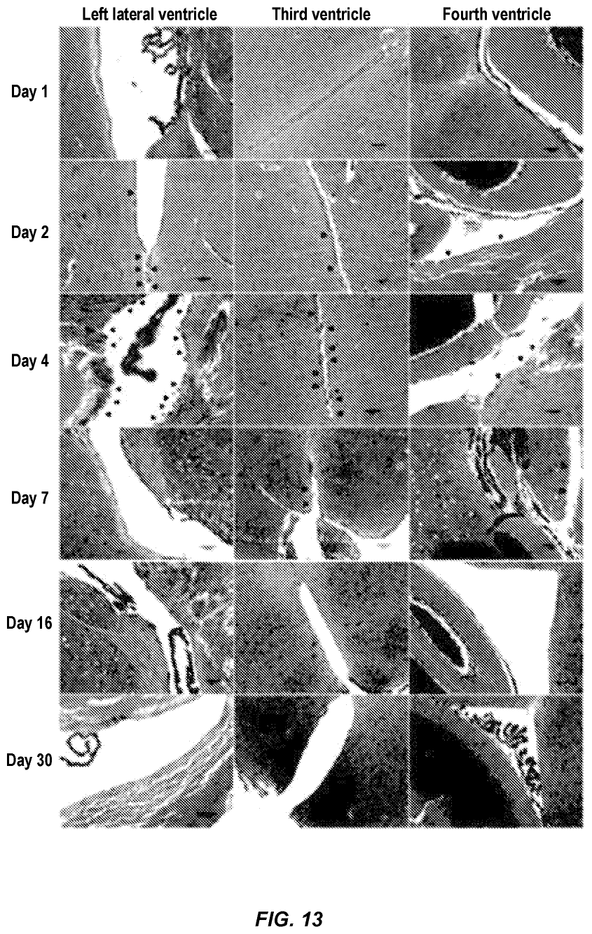

CBA/J mice were injected IVT with 1×10⁷ PFU of G207, radiation-inactivated G207, saline, or 10% glycerol (vehicle). Mice in the inactivated virus, saline, and 10% glycerol groups displayed no toxicity; however, 3 of 10 or 30% of the G207 mice became morbid, requiring euthanasia 4 days after IVT delivery. These data suggest that toxicity from IVT G207 is unrelated to the vehicle or viral antigen(s) alone. To further characterize the nature of the toxicity displayed and the extent of the inflammatory response, we repeated the study outline above, sacrificed the mice on days 1, 2, 3, 4, 7, 16 and 30 and performed IHC. The ependymal lining within the ventricles of the brain appeared normal on day 1; however, by day 2, there was focal disruption of the ependymal lining with evidence of nuclear shrinkage, fragmentation, and cell death (FIG. 1). Of note, the ependymal cells examined stained positive for HSV-1 on day 2 and by day 3, there was more diffuse disruption of the ependymal lining and noted edema; on day 4 there was diffuse disruption of the ependymal lining with minimal to no normal lining having been observed. Inflammatory infiltrates were noted on days 4 and 7. Similar findings were seen in the left lateral, third, and fourth ventricles (FIG. 13) whereas the ependymal lining was normal in mice that received inactivated oHSV (FIG. 8A). The ependymal cells stained positive for HSV-1 and late viral protein gD was also present in the ependymal lining on day 2 (FIG. 1C) suggesting that such damage may be due to a productive viral infection. Between days 16-30, there was evidence of a slow repair of the ependyma with no residual inflammation; however focal areas of ependymal lining loss remained (FIGS. 1B and 13).

### Damage to the Ependymal Lining is in Part CD8⁺ T-Cell Mediated

To determine if the productive infection noted above resulted in immune cell infiltration, IHC was performed, staining for CD8, using brain tissue from mice sacrificed on days 3 and 4 post IVT G207 (FIG. 1D). The results obtained showed that inactivated IVT delivered G207 resulted in no immune cell infiltration (FIG. 8A), whereas IVT G207 did in fact display CD8⁺ T-cell peri-ventricular infiltration. Based on these results, it was hypothesized that CD8⁺ cytotoxic T-cells were causing focal inflammation thereby amplifying damage to the ependymal lining after IVT G207. To test this, circulating CD8⁺ cells in the blood and mobilized from lymphoid tissues were depleted by treatment twice a week with a cytotoxic anti-CD8 antibody. The results showed significant reduction of CD8+ cells in CBA/J mice by flow cytometry after IP injection of anti-CD8 antibody but not by treatment with an isotype immunoglobulin control. Flow cytometry confirmed selective reduction of CD8+ cells and not CD4+ cells (FIG. 8B). IVT oHSV toxicity in mice was compared in separate groups of mice with and without CD8+ cells depleted. It was determined that mice without CD8+ cells (due to cytotoxic antibody) lost less weight after IVT oHSV than mice with normal CD8+ on day 4 and 7 (FIG. 1E). Mice with functional CD8+ T cells lost 1.2-fold and 1.4-fold more weight than mice depleted of CD8+ T cells at days 4 and 7 post-treatment. This was consistent with our IHC staining data showing CD8+ cells infiltrating the ependymal lining starting on day 3. The result indicates that CD8+ cells likely contribute to IVT oHSV toxicity.

### Low Dose G207 Prevents Ependymal Damage Related to Subsequent Therapeutic G207 IVT Delivery

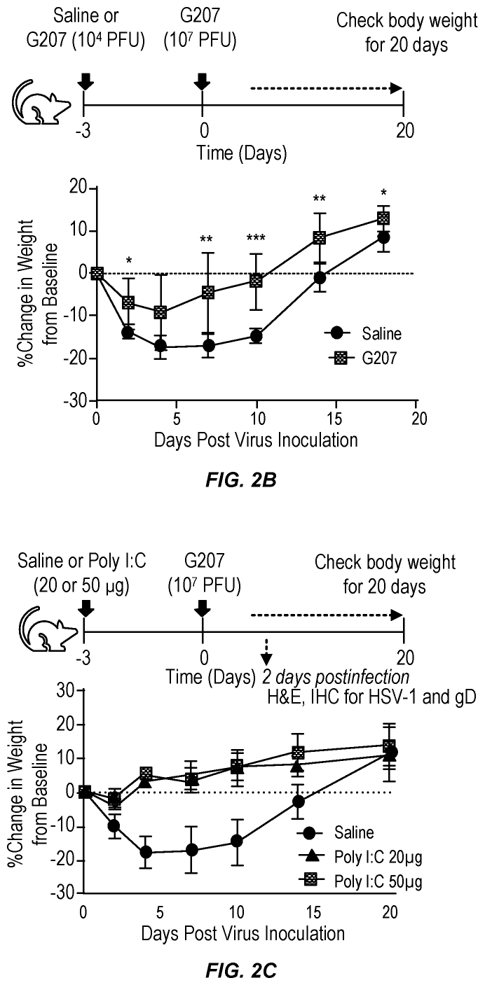

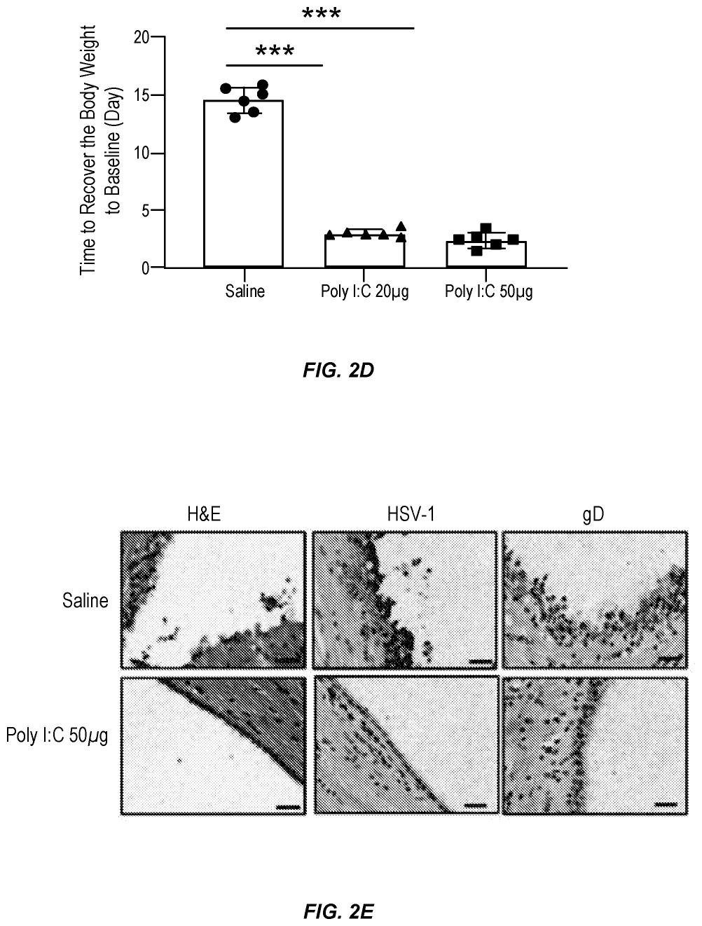

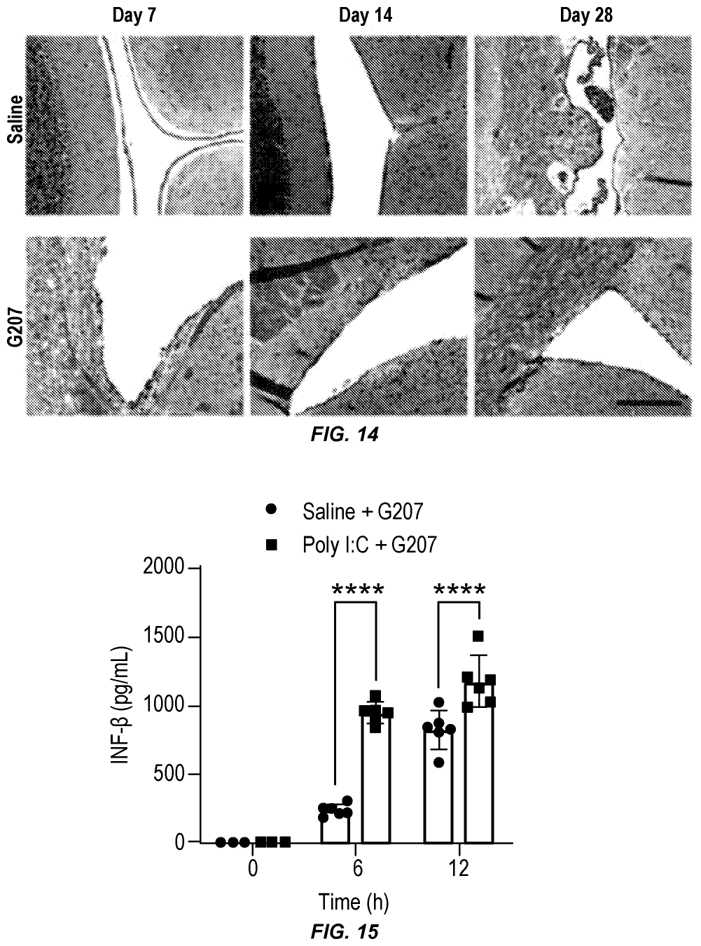

To determine if subsequent 1×10⁷ PFU doses of IVT oHSV caused similar toxicity as the first dose, mice were retreated with a second and third dose after recovery of body weight to baseline and changes in weight were assessed. Subsequent doses of G207 did not appear to result in additional toxicity in the oHSV-sensitive CBA/J mice, using weight loss as a surrogate measure (FIG. 2A). While the ependymal lining showed areas of disruption a week after the first IVT oHSV dose, little to no additional toxicity was seen with each successive dose (FIG. 14). These data suggest that each subsequent dose of IVT oHSV provided a protective effect from additional toxicity. This indicated the first dose resulted in an interferon (IFN)-induced antiviral response within the ependymal cells; IFN is known to prevent productive HSV infections by activating the PKR pathway, inducing eIF2α which ultimately shuts down protein translation in normal cells (He B, Gross M, Roizman B. The gamma(1)34.5 protein of herpes simplex virus 1 complexes with protein phosphatase 1 alpha to dephosphorylate the alpha subunit of the eukaryotic translation initiation factor 2 and preclude the shutoff of protein synthesis by double-stranded RNA-activated protein kinase. Proc Natl Acad Sci USA. 1997 Feb. 4; 94(3):843-8. doi: 10.1073/pnas.94.3.843.PMID: 9023344).

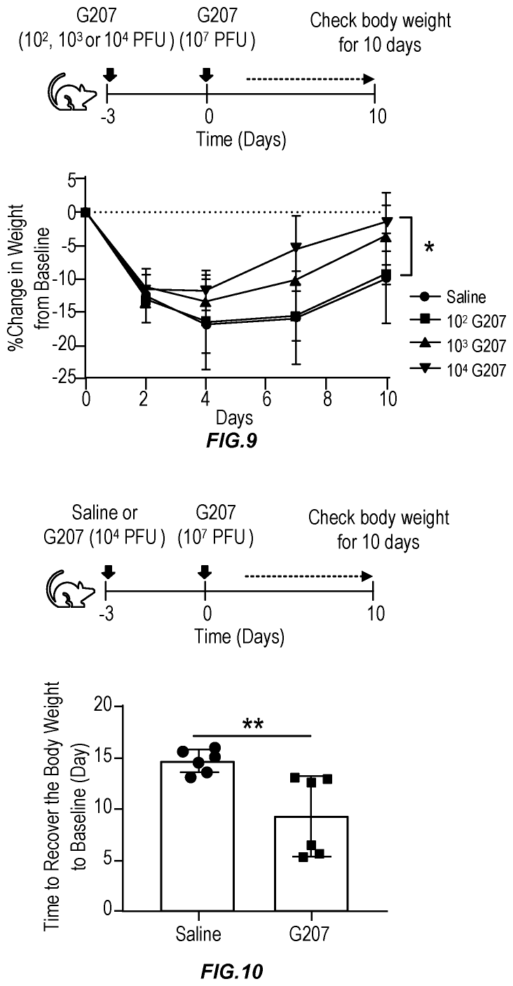

Given this, it was hypothesized that ependymal cells could be “primed” via interferon-induction thereby protecting them prior to IVT G207. Since it was determined that 1×10⁷ PFU of G207 caused toxicity, lower doses of virus (i.e., 10², 10³, or 10⁴ PFU) were tested as preconditioning modalities for mitigation of IVT related toxicity (FIG. 9). Importantly, a low dose of IVT G207 (10⁴ PFU) followed by 10⁷ PFU of IVT G207 resulted in less weight loss and a shorter return to baseline weight as compared to the saline control (FIGS. 2B and 10). These data show that pretreatment with low-dose G207 prior to high-dose G207 mitigates IVT related toxicity. Seven (7) days after the standard treatment dose, mice pretreated with saline lost 16.9±2.7% of their pretreatment body weight, whereas mice pretreated with low-dose oHSV lost only 4.4±9.2% of their weight (P=0.0002). By day 10, saline-pretreated mice lost 14.8±1.8% of their pretreatment body weight, whereas mice that received low-dose oHSV had nearly recovered their baseline weight (1.8±6.5%; P<0.0001). Furthermore, saline-pretreated mice took approximately 1.5-fold longer to recover their weight back to baseline (FIG. 10). Taken together, these data indicate that a single low dose of IVT oHSV provides a protective effect and is capable of mitigating toxicity from a subsequent standard IVT treatment dose.

### Polyinosinic-Polycytidylic Acid (Poly [I:C]) Prevents Ependymal Damage Secondary to IVT Delivery of G207

Given the findings presented above, other methods of limiting IVT related toxicity by IFN induction prior to the delivery of a therapeutic G207 dose were explored. Poly(I:C), a toll-like receptor (TLR)-3 agonist and known inducer of IFN expression and signaling (Matsumoto M, Seya T. TLR3: interferon induction by double-stranded RNA including poly(I:C). Adv Drug Deliv Rev. 2008 Apr. 29; 60(7):805-12. doi: 10.1016/j.addr.2007.11.005. PMID: 18262679), was used prior to the delivery of a high dose (i.e., 1×10⁷ PFU) IVT G207. Administration of IVT poly(I:C) 72 hours prior to IVT oHSV resulted in significantly less toxicity as evidenced by a decrease in weight loss and a shorter time to recovery in body weight mimicking the effects seen with low-dose (i.e. 1×10⁴) G207 pre-treatment (FIGS. 2C and 2D). Mice pretreated with poly I:C only lost 1.5±2.6% of body weight two days after the treatment dose of oHSV and were above their baseline body weight by day 4, whereas saline-pretreated mice lost 17.6±4.7% of their body weight by day 4. Of note, the protective effect of poly(I:C) did not appear to be dose-dependent, at least within certain ranges, as no difference was seen between 20 μg and 50 μg (FIG. 2D). Furthermore, IHC of the ependymal lining revealed a normal appearance in cellular architecture in those mice that received poly(I:C) prior to G207 but not in those control mice that received saline prior to G207. Critically, evidence of a productive viral infection was present in the saline control group but not in the poly(I:C) pretreatment group (FIG. 2E).

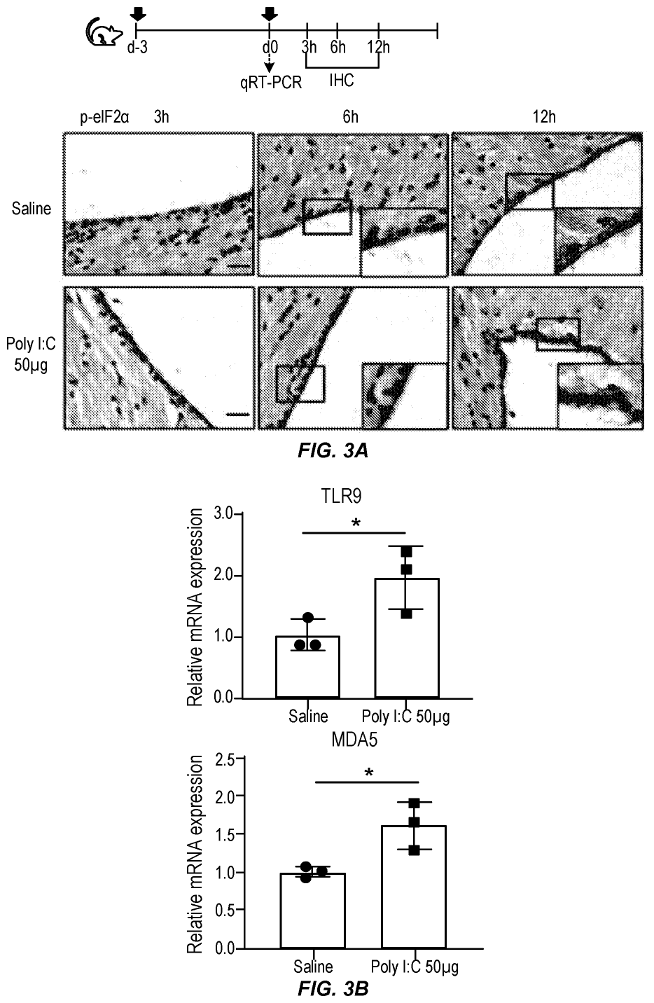

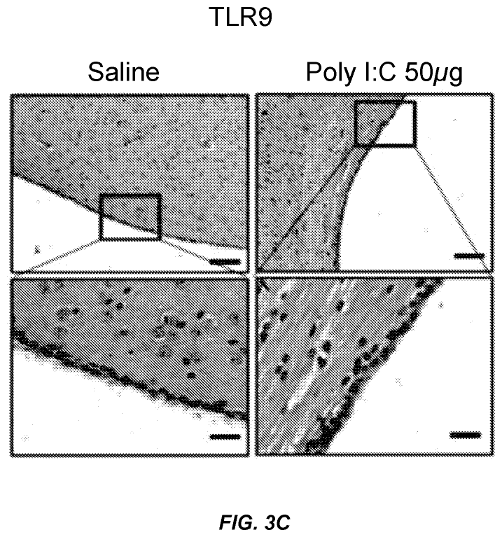

To investigate additional mechanisms underlying the protective effects noted after poly(I:C) pretreatment, phosphorylated eIF2α (p-eIF2a), a core component in host anti-viral responses (Lussignol M, et al. J Virol. 2013; 87(2):859-71), was compared in mice receiving IVT saline or 50 μg poly(I:C) prior to IVT G207. IHC staining with p-eIF2α antibody was performed at 3, 6, or 12 hours post-IVT delivery. p-eIF2α staining was detected after 6 hours in the poly(I:C) treated groups and after 12 hours in the saline treated controls group (FIG. 3A). These data show that the virus replication is inhibited earlier in the ependymal cells of those mice that were pre-treated with poly(I:C). To further elucidate the differences noted in eIF2α activation, the relative mRNA expression level of two cellular pattern recognition receptors, TLR9 and MDA5 (Melanoma Differentiation-Associated protein 5) was compared at day 3 in the mice receiving IVT saline or poly(I:C). The mRNA expression level of TLR9 and MDA5 in poly(I:C) treated-group was higher than in saline-treated control (FIG. 3B). Additionally, IHC staining of TLR9 matched the mRNA expression level (FIG. 3C). Therefore, increase of TLR 9 expression is a likely cause for increased IFN expression in HSV-infected ependymal cells. Furthermore, activation of PKR/eIF2α signaling pathway in poly I:C-treated group is faster than in saline-treated group. These data together show that poly I:C can prevent damage of ependymal cell lining and decrease toxicity.

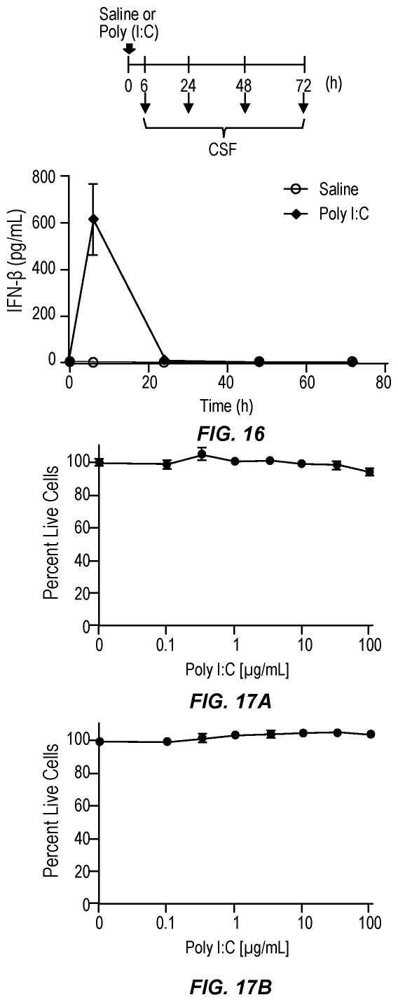

To characterize protection of the ependyma through IFN induction and subsequent phosphorylation of eIF2α, the amount of IFN-β was determined in the CSF after a protective dose of IVT poly I:C or saline and then a treatment dose of IVT oHSV. IFN-β was detected 6 hours after the pretreatment dose but was undetectable by 48 hours and prior to the oHSV treatment dose (FIGS. 15 and 16). Importantly, a significant increase in IFN-β was seen at 6 and 12 hours after oHSV in mice pretreated with poly I:C compared to saline. Staining of p-eIF2α, which mediates translation shutoff, was detected after 6 and 12 hours in poly I:C-pretreated mice but not in saline-pretreated mice (FIG. 3A). Taken together, these data indicate that pretreatment with IVT poly I:C prevents damage to ependymal cells from subsequent IVT oHSV. Further, these data show that pretreatment acts to “prime” ependymal cells by inhibiting viral replication earlier via phosphorylation of eIF2α.

### IVT G207 Demonstrates Efficacy in Preclinical Models of Pediatric Medulloblastoma

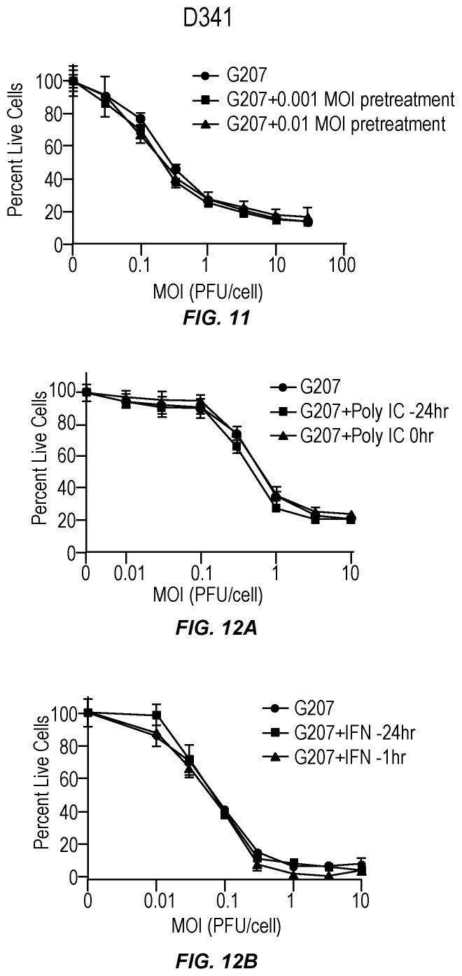

Low-dose G207 (10⁴ PFU) pre-treatment with high-dose G207 (10⁷ PFU) to examine the potential therapeutic efficacy of such an IVT approach in tumor-bearing mice. To confirm pre-conditioning with low dose G207 prior to the delivery of high dose G207 did not perturb the ability of our oHSV to effectively kill tumor cells, a cytotoxicity assay was performed using D341-luc cells in which we demonstrated that the LD₅₀ of two different low-dose pretreated cells groups was the same as non-pretreated groups (FIG. 11), indicating that pretreatment with low dose G207 does not affect its ability to kill medulloblastoma cells.

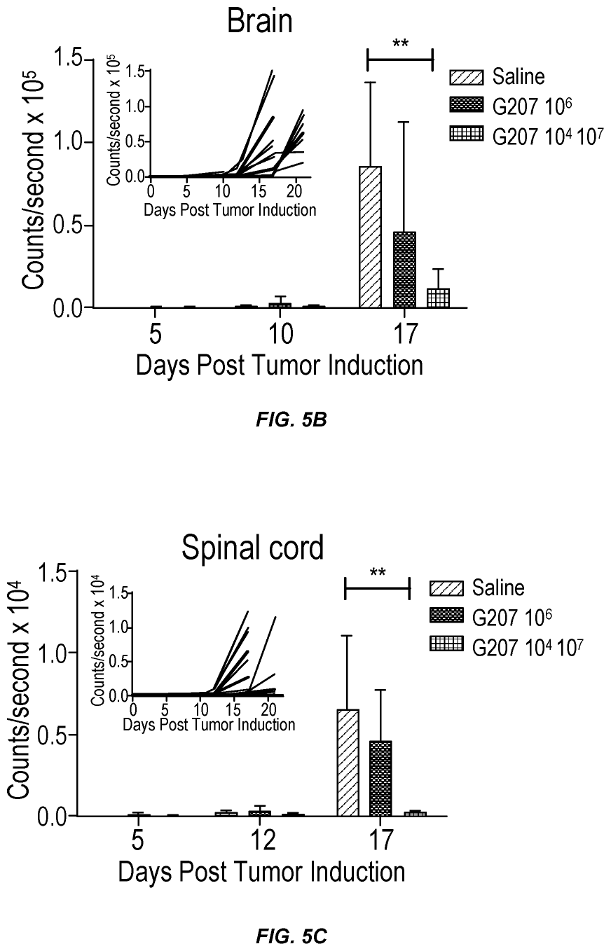

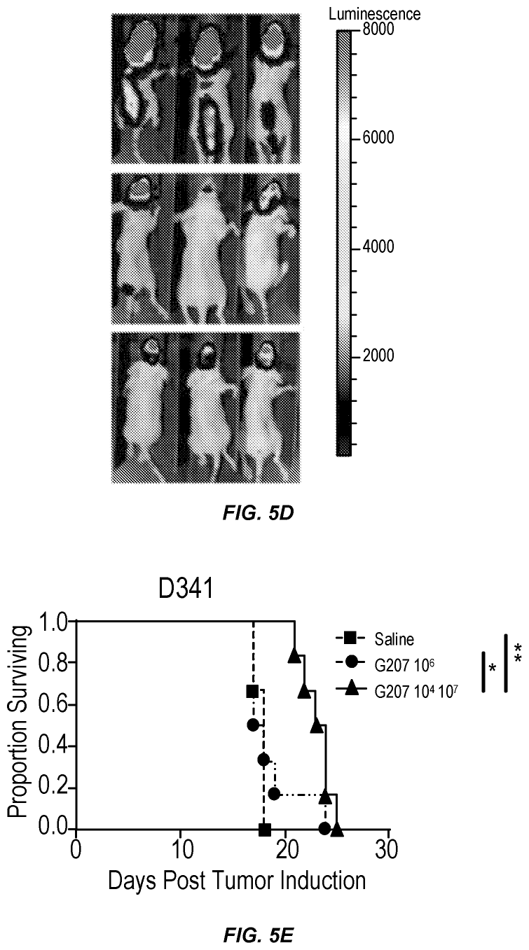

Athymic nude mice underwent IVT injection of D341-luc cells and 72 hours later received IVT saline or low dose (10⁴ PFU) G207. Three and 10 days later mice received IVT injections of saline, 1×10⁶ or 1×10⁷ PFU of G207. In line with our previous studies, mice receiving IVT low-dose G207 prior to high-dose virus displayed significantly less weight loss. Bioluminescence data showed that the tumor signal in both brain and spinal cord in the pre-treated high-dose G207 group was lower than the other groups (FIG. 5B-D). Critically, survival within this group was significantly prolonged survival as compared to control (FIG. 5E).

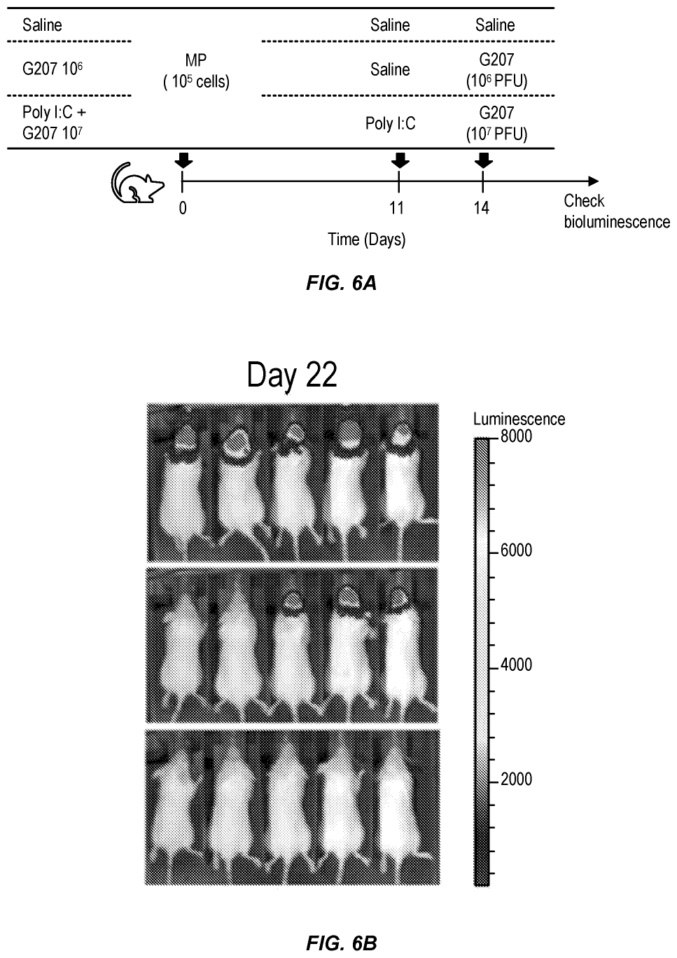

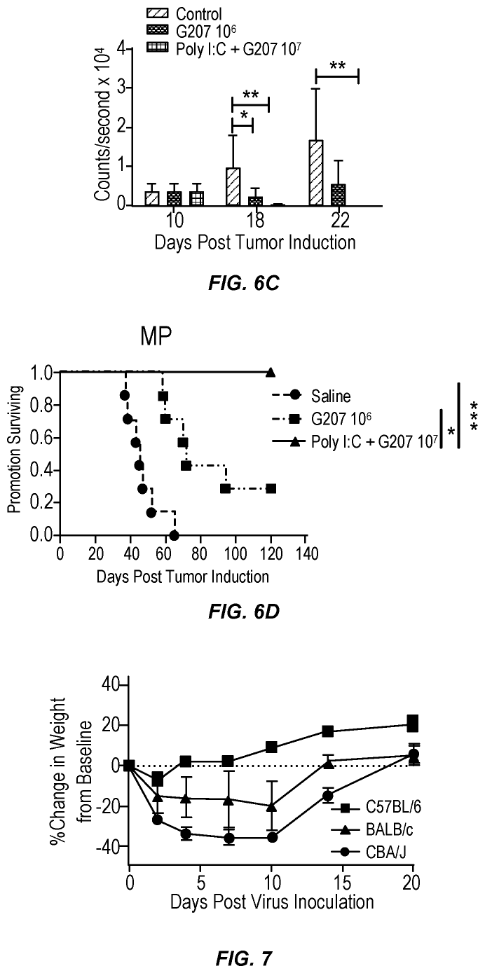

Poly(I:C) pretreatment with high-dose G207 (10⁷ PFU) was tested in an effort to establish the preclinical efficacy of such an approach in immunocompetent mouse model (FIG. 6A-D). Albino B6 mice were inoculated IVT with MP tumor cells expressing Myc and a dominant-negative form of p53 and 11 days later mice received IVT saline or poly(I:C). Three days later mice received IVT injection of saline, 10⁶ or 10⁷ PFU of G207. Mice treated with saline had a median survival time of 40 days. IVT 10⁶ G207 had a modest effect on tumor growth (median survival, 72 d). The pretreated Poly I:C followed IVT 10⁷ G207 markedly inhibited tumor growth leading to tumor-free survival in 100% of mice (FIG. 6D). These results suggested that Poly I:C pretreatment could also prevent toxicity in the immunocompetent mouse model. Taken together, the delivery of IVT G207 showed efficacy in both immunocompromised and immunocompetent mouse models of medulloblastoma.

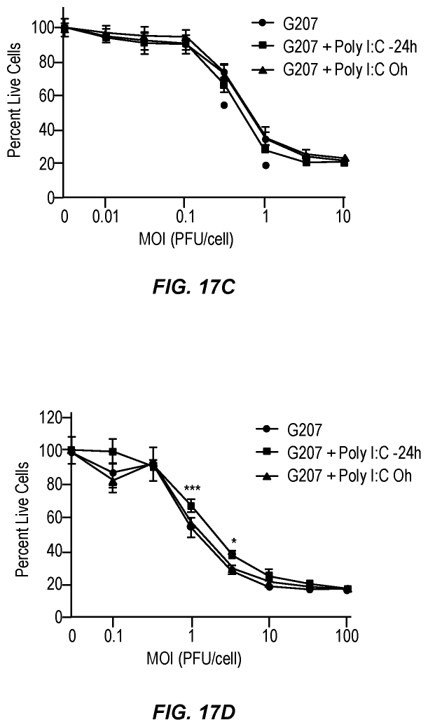

IVT G207 was tested in an immunocompetent murine model of disseminated MYC-driven, p53-mutant medulloblastoma (MP) using a protective dose of poly I:C pre-G207 treatment after confirming in vitro that poly I:C did not result in a treatment effect when given alone nor did it impact G207 cytotoxicity (FIGS. 17A, 17B, 17C, and 17D). Albino B6 mice were injected with 1×10⁵ MP tumor cells, which disseminated locally in the brain. Eleven days after tumor induction, mice received IVT saline or poly I:C, followed three days later by IVT saline or a single 1×10⁷ PFU G207 dose. The later time point for treating with G207 in this model was due to different tumor growth kinetics as assessed by bioluminescence imaging. After treatment, bioluminescence disappeared in G207-treated mice but continued to increase in controls (FIGS. 6B and 6C). Saline-treated mice had a median survival of 40 days, whereas G207-treated mice survived for >120 days without signs of brain tumors (FIG. 6D). Consistent with prior reports, a single dose of poly I:C alone provided no survival benefit compared to saline control (Figure S9). These results indicate that IVT G207 effectively targets aggressive disseminated human and murine models of medulloblastoma.

IVT Delivery of G207 Prevents Development of Metastatic Disease within the Spinal Cord

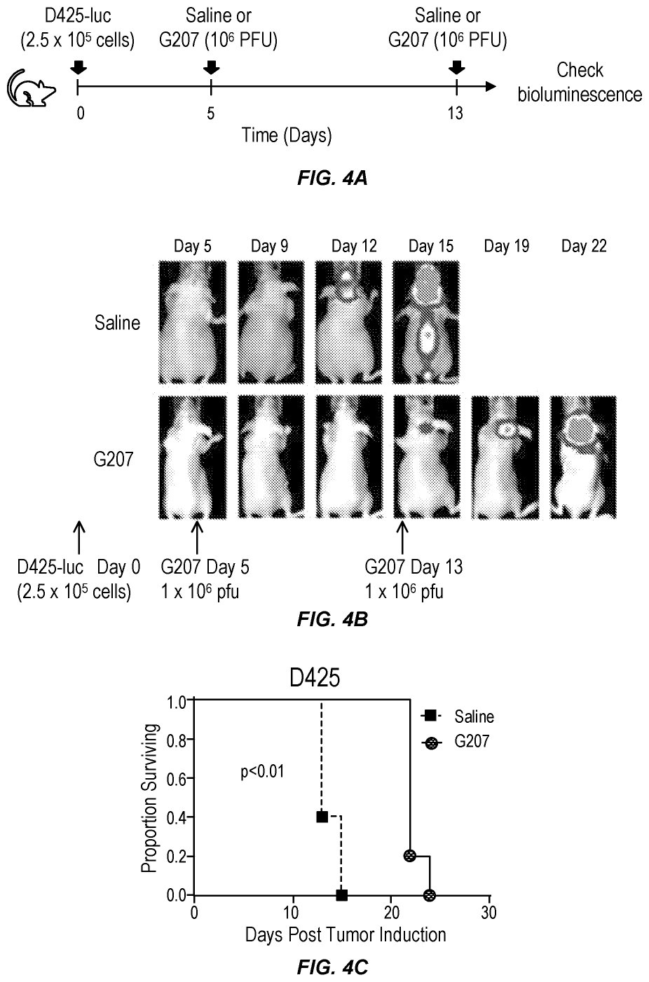

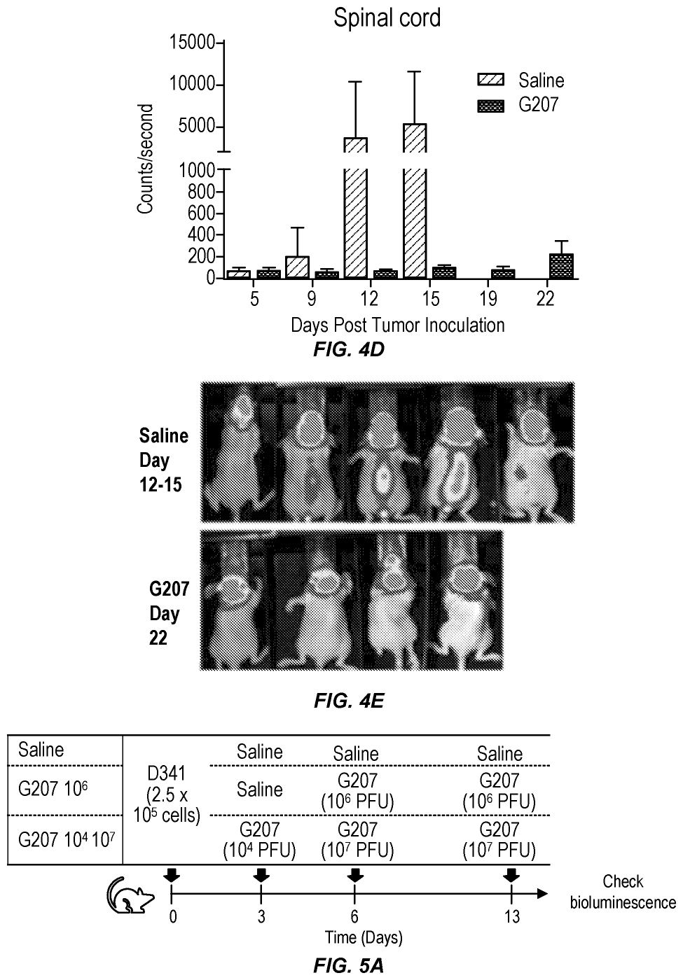

To test the efficacy of IVT oHSV, a metastatic model was developed by seeding 2.5×10⁵ D425-luc cells, a pediatric patient-derived Group 3 medulloblastoma xenograft, via IVT injection in athymic nude mice. The mice developed both primary tumors and tumors spread throughout the spinal column which were tracked with bi-weekly imaging. Five days after tumor induction, 1×10⁶ PFU of G207 or saline as a control were administered. This 10-fold lower dose than was used for the toxicity studies was chosen to see if the virus could target metastatic disease effectively at a lower dose. The single G207 dose significantly prolonged survival by 50% (p=0.008) and reduced spinal metastases. To confirm the results of the study and to establish the safety and efficacy of repeat dosing of virus, the experiment was repeated with 2.5×10⁵ D425-luc cells and followed with a second dose 8 days later. The two doses of virus were safe and significantly slowed primary tumor growth and prevented spinal disease (FIGS. 4B and C). The treatment prolonged median survival from 13 to 22 days (p<0.001) (FIG. 4D). These findings indicated that IVT G207 can prolong survival and decrease spinal metastases at a 10-fold lower dose than is typically used in intratumoral injections.

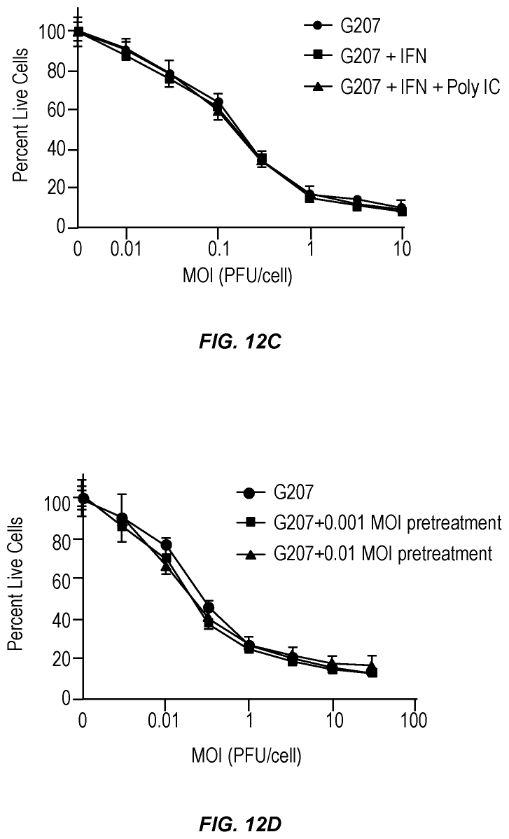

FIGS. 12A-12D shows effect of Poly IC and IFN on G207 cytotoxicity with pediatric brain xenograft model D341 and D425. FIG. 1A is a graph showing D341 pediatric brain tumor xenograft cells treated with G207 and/or Poly-IC. FIG. 1B is a graph showing D341 pediatric brain tumor xenograft cells treated with G207 and/or interferon (IFN). FIG. 1C is a graph showing D425 pediatric brain xenograft cells treated with G207, G207 and IFN or G207 and IFN and Poly-IC. The cells were plated and incubated for 24 hrs. Poly IC (2 μg), IFN (200 IU) or Poly IC and IFN were added to the wells prior to viral inoculation. G207 was added at increasing MOI. At 72 hrs after virus inoculation, alamarBlue assay was performed to evaluate cytotoxicity with IC50. FIG. 12D is a graph showing effect of low-dose pretreatment on G207. D425, pediatric brain tumor xenograft cell was treated with low-dose G207 [0.01 or 0.001 MOI (PFU/cell)]. At 72 hrs later, the cells were plated and incubated for 24 hrs. G207 was added at increasing MOI. At 72 hrs after viral inoculation, alamarBlue assay was performed to evaluate cytotoxicity with IC50. The viral replication and killing of cells was not perturbed by the preconditioning agents employed.

In summary, toxicity from IVT oHSV can be sufficiently mitigated with an IVT low dose of oHSV or IFN-inducing agent poly I:C to enable safe administration of therapeutic dose(s) of IVT oHSV. Moreover, this protective strategy enabled safe delivery of multiple treatment doses of IVT G207 and prolonged survival in human and murine disseminated medulloblastoma models. These findings indicate that toxicity from IVT oHSV can be sufficiently alleviated for therapeutic effect and support clinical translation of IVT or intrathecal G207.

1. A method of treating a central nervous system cancer in a subject comprising
(a) administering to the subject a preconditioning agent, and,
(b) subsequent to step (a), administering to the subject a therapeutic dose of an oncolytic virus,
wherein the administration steps of (a) and (b) comprise administration to the subject's cerebrospinal fluid or periependymal region to treat the central nervous system cancer in the subject.

2. The method of claim 1, wherein the preconditioning agent is an immunostimulant.

3. The method of claim 1, wherein the preconditioning agent induces a transient anti-viral response and/or upregulates an interferon response.

4. The method of claim 1, wherein the immunostimulant is selected from the group consisting of a toll-like receptor agonist, a subtherapeutic dose of an oncolytic virus, and/or an interferon.

5. The method of claim 4, wherein the immunostimulant is a toll-like receptor agonist.

6. The method of claim 5, wherein the toll-like receptor agonist is Poly-IC, Poly-IC:LC, or a fragment of a repetitive unmethylated CpG nucleic acid sequences.

7. The method of claim 4, wherein the preconditioning agent is a subtherapeutic dose of an oncolytic virus.

8. The method of claim 7, wherein the subtherapeutic dose of the oncolytic virus is at least 1×10¹ PFUs less than the therapeutic dose of the oncolytic virus.

9. The method of claim 7, wherein the oncolytic virus administered as the preconditioning agent is the same oncolytic virus administered at the therapeutic dose.

10. The method of claim 1, wherein the immunostimulant is administered prior to administration of the therapeutic dose of the oncolytic virus.

11. The method of claim 1, wherein the oncolytic virus is a replication-competent, recombinant herpes simplex virus-1 (HSV-1).

12. The method of claim 11, wherein the HSV-1 comprises a mutation in γ₁34.5 gene and a mutation in ICP6 gene.

13. The method of claim 12, wherein the mutation in the ICP6 gene comprises an insertion of the E. coli lacZ gene.

14. The method of claim 11, wherein the therapeutic dose of HSV-1 is 10³ to 10¹⁵ PFU.

15. The method of claim 1, wherein the administration of step (a) is intraventricular, wherein the administration of step (b) is intraventricular, or wherein both administration steps are intraventricular.

16. The method of claim 1, wherein the administration of step (a) is into the subarachnoid space, wherein the administration of step (b) is into the subarachnoid space, or wherein both administration steps are into the subarachnoid space.

17. The method of claim 1, wherein the administration of step (a) is into a peri-ependymal region, wherein the administration of step (b) is into the peri-ependymal region, or wherein both administration steps are into a the peri-ependymal region.

18. The method of claim 1, further comprising administering one or more additional therapeutic doses of the oncolytic virus.

19. The method of claim 1, further comprising screen for tumor growth or metastasis.

20. The method of claim 1, further comprising administering one or more therapeutic agents to the subject.

21. The method of claim 20, wherein the therapeutic agent is a chemotherapeutic agent.

22. The method of claim 1, wherein the central nervous system cancer is a drop metastasis or a leptomeningeal disease.

23. The method of claim 1, wherein the preconditioning agent protects ependymal cells.
